(19) United States
(12) Patent Application Publication
(10) Pub. No.: US 2010/0280070 A1
(43) Pub. Date: Nov. 4, 2010

(54) NOVEL TETRAHYDROQUINOLINES AS AROMATASE INHIBITORS

(76) Inventor: Raghuram Rao AKKINEPALLY, CHANDIGARH (IN)

Correspondence Address: JEROME D. JACKSON (JACKSON PATENT LAW OFFICE), 211 N. UNION STREET, SUITE 100, ALEXANDRIA, VA, 22314, US

(21) Appl. No.: 12/810,808

(22) PCT Filed: Jan. 2, 2009
(86) PCT No.: PCT/IN09/00002

§ 371 Date: Jun. 25, 2010

(30) Foreign Application Priority Data
Jan. 4, 2008 (IN) 41/DEL/2008

(51) Int. Cl. A61K 31/47; C07D 215/233; C07D 401/04; A61K 31/4709; C07D 401/06; A61P 35/00

(52) U.S. Cl. 514/312; 546/153; 546/162; 514/313

(57) ABSTRACT

The invention relates to synthesis and biological screening of novel tetrahydroquinolines of formula (I), their derivatives, their stereoisomers, their pharmaceutically acceptable salts and pharmaceutically acceptable compositions containing them for aromatase inhibition: (I). The present invention also relates to a process for the preparation of the novel tetrahydroquinolines, their derivatives, their stereoisomers, their pharmaceutically acceptable salts and pharmaceutically acceptable compositions containing them. These compounds are useful in for aromatase inhibition, particularly in the treatment and/or prevention of cancer, particularly breast cancer, more particularly hormone dependent breast cancer.

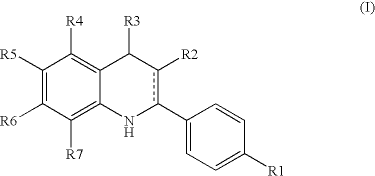

### FIELD OF INVENTION

The invention relates to synthesis and biological screening of novel tetrahydroquinolines for aromatase inhibition. The invention also relates to the methods of preparation of the novel tetrahydroquinolines.

### BACKGROUND OF THE INVENTION

Aromatase is a microsomal cytochrome P450 enzyme belonging to CYP 19 super family. It is involved in the specific conversion of androgens to estrogens. In postmenopausal women the main sites of aromatisation are skin, adipose tissue and breast. Aromatase localized in breast tumor produces sufficient estrogen for its proliferation. Hence it is an important target for the treatment of hormone dependent breast cancer in postmenopausal women. The third generation aromatase inhibitors are approved for the treatment of postmenopausal women with metastatic estrogen dependent breast cancer. Current clinical studies of aromatase inhibitors are focusing on the use of these agents in the adjuvant, neoadjuvant and chemoprevention settings for the treatment of breast cancer.

Importance of aromatase inhibitors is not only confined to treatment and prevention of hormone dependant breast cancer but also to pathological conditions in which estrogen plays pivotal role. The short-term application letrozole has recently been successful for the induction of ovulation in women with infertility. If proved, the use of aromatase inhibitors in premenopausal women. It opens the possibility of using aromatase inhibitors to treat benign breast conditions such as cyclic breast pain, fibroadenomata and recurrent cystic breast disease. Estrogen and its metabolites also related to their role in the disorders in male population. Although rare male breast cancer also exists. Leydig cell tumors are induced by aromatase over expression. Pre clinical studies have suggested that estrogen may have role in the development and progression of prostatic cancer and benign hyperplasia. There are other estrogen dependent non tumorogenic conditions like gynocomestia.

Quinolines are an important class of compounds in the field of pharmaceuticals and exhibit a wide spectrum of biological activity. These compounds are reported to display inhibition of osteoporosis, estrogen dependent breast cancer, endometriosis and uterine fibrosis by binding to estrogen receptors and by several other mechanisms of action. By virtue of their modulation of nuclear receptors like liver-X and retenoid-X receptors they can prevent dyslipidemia, disorders related to cholesterol and bile acid metabolism, proliferative vitreoretinopathy (PVR), retinal detachment and dry eye. Apart from these indications they are known to inhibit cholesteryl ester transfer protein (CETP) and modulate signal transducer and activator of transcription (STAT 6) pathway.

### Comparison of Existing Aromatase Inhibitors:

| | IC₅₀ from human placental |
|---|---|
| Compound | microsomal assay (μM) |
| Quinoline derivatives | No report |
| Therapeutic agents | |
| Aminoglutethimide | 1.90 |
| Letrozole | 0.011 |
| Anasatrozole | 0.023 |
| Fadrozole | 0.005 |
| Formestane | 0.050 |
| Exemestane | 0.050 |
| Flavanoid derivatives | |
| Flavone | 8.0 |
| 7-Hydroxyflavone | 1.0 |
| Chrysin | 0.5 |
| Apigenin | 2.9 |
| Naringenin | 9.2 |

| | IC₅₀ from choriocarcinoma |
|---|---|
| Compound | cell line assay (μM) |
| Quinoline derivatives | No report |
| Therapeutic agents | |
| Aminoglutethimide | 15.80 |
| Letrozole | 0.070 |
| Anasatrozole | 0.990 |
| Fadrozole | 0.070 |
| Flavanoid derivatives | |
| Flavone | >100 |
| 7-Hydroxyflavone | 0.35 |
| Chrysin | 0.5 |
| Apigenin | 0.18 |
| Naringenin | 1.4 |

| | Breast cancer | | Mammary fibroblast | |
|---|---|---|---|---|
| | homogenates | | Cultures | |
| Compound | IC₅₀ (nM) | RP | IC₅₀ (nM) | RP |
| Aminoglutethimide | 4500 | 1 | 8000 | 1 |
| Anastrozole | 10 | 450 | 14 | 570 |
| Letrozole | 2.5 | 1800 | 0.8 | 10,000 |
| Formestane | 30 | 150 | 45 | 180 |
| Exemestane | 15 | 300 | 5 | 1600 |

Quinolines are least explored compounds for aromatase inhibition. To the best of our knowledge there has been no medicinal chemistry effort to develop quinoline based aromatase inhibitors. Present invention deals with the synthesis and biological screening of novel tetrahydroquinolines for aromatase inhibition.

### OBJECTS OF THE INVENTION

The primary object of the invention is to provide for novel tetrahydroquinolines compounds for the inhibition of aromatase enzyme.

Another object of the invention is to provide for a method for the preparation of the novel tetrahydroquinolines compounds of the invention.

Yet another object of the invention is to provide for a method using the tetrahydroquinolines compounds in the treatment of cancer, particularly in the treatment of breast cancer, more particularly but not limited to hormone dependent breast cancer.

### SUMMARY OF THE INVENTION

The present invention relates to a compound of formula (I):

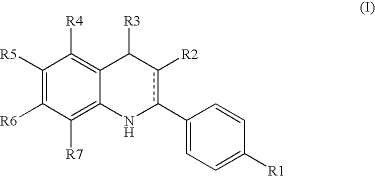

wherein, dotted line represents optional double bond;
R1 is —H, halo, —(C₁-C₃)alkyl, —O(C₁-C₃)alkyl or —CN; R2 is —H or

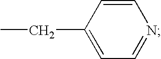

R3 comprises —O—, heterocycle moiety, preferably of ring size 5-6; wherein the ring is selected from imidazole, triazole, tetrazole and/or pyridine; wherein further the said ring comprises substituents selected from —H, —(C₁-C₃)alkyl, —O(C₁-C₃)alkyl, F, —CF₃, —NH₂, —N(CH₃), —N(CH₃)₂, —SH, —SCH₃, —SCH₂CH₃ and/or combinations thereof;
R4, R5, R6, R7 are, independently a moiety selected from the group consisting of —H, halo, —(C₁-C₃)alkyl, —O(C₁-C₃)alkyl or —CF₃;
or a pharmaceutically acceptable salt or derivative thereof.

In a preferred embodiment, R1 in compound of formula (I) is preferably selected from the group comprising —H, Cl, Br, F, —CH₃, —OCH₃ or —CN.

In another preferred embodiment, R3 in compound of formula (I) is preferably selected from the group comprising

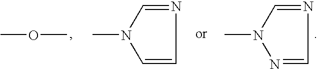

In yet another preferred embodiment, R3 in compound of formula (I) is heterocyclic ring, preferably a five membered ring comprising 1-3 nitrogen atoms in the ring.

In a preferred embodiment, R4, R5, R6 and R7 in compound of formula (I) are preferably selected from H or Br.

In a preferred embodiment, the compound of formula (I) is selected from the group comprising:
2-Phenyl-4-imidazol-1-yl-1,2,3,4-tetrahydro-quinoline;
2-(p-Tolyl)-4-imidazol-1-yl-1,2,3,4-tetrahydro-quinoline;
2-(4-Methoxy phenyl)-4-imidazol-1-yl-1,2,3,4-tetrahydro-quinoline;
2-(4-Chloro phenyl)-4-imidazol-1-yl-1,2,3,4-tetrahydro-quinoline;
2-(4-Bromo phenyl)-4-imidazol-1-yl-1,2,3,4-tetrahydro-quinoline;
4-(4-Imidazol-1-yl-1,2,3,4-tetrahydro-quinolin-2-yl)-benzonitrile;
2-(4-Fluoro phenyl)-4-imidazol-1-yl-1,2,3,4-tetrahydro-quinoline;
2-Phenyl-4-[1,2,4]triazol-1-yl-1,2,3,4-tetrahydro-quinoline;
2-(p-Tolyl)-4-[1,2,4]triazol-1-yl-1,2,3,4-tetrahydro-quinoline;
2-(4-Methoxy phenyl)-4-[1,2,4]triazol-1-yl-1,2,3,4-tetrahydro-quinoline;
2-(4-Chloro phenyl)-4-[1,2,4]triazol-1-yl-1,2,3,4-tetrahydro-quinoline;
2-(4-Bromo phenyl)-4-[1,2,4]triazol-1-yl-1,2,3,4-tetrahydro-quinoline;
4-(4-[1,2,4]Triazol-1-yl-1,2,3,4-tetrahydro-quinolin-2-yl)-benzonitrile;
2-(4-Fluoro phenyl)-4-[1,2,4]triazol-1-yl-1,2,3,4-tetrahydro-quinoline;
2-Phenyl-3-((pyridin-4-yl)methyl)quinolin-4(1H)-one;
2-(p-Tolyl)-3-((pyridin-4-yl)methyl)quinolin-4(1H)-one;
2-(4-Methoxy phenyl)-3-((pyridin-4-yl)methyl)quinolin-4(1H)-one;
2-(4-Chloro phenyl)-3-((pyridin-4-yl)methyl)quinolin-4(1H)-one;
2-(4-Bromo phenyl)-3-((pyridin-4-yl)methyl)quinolin-4(1H)-one;
4-(4-oxo-3-((pyridin-4-yl)methylene)quinolin-2-yl)benzonitrile;
2-(4-Fluoro phenyl)-3-((pyridin-4-yl)methyl)quinolin-4(1H)-one;
6,8-Dibromo-2-phenyl-3-((pyridin-4-yl)methyl)quinolin-4(1H)-one;
6,8-Dibromo-2-(p-tolyl)-3-((pyridin-4-yl)methyl)quinolin-4(1H)-one;
6,8-Dibromo-2-(4-Methoxy phenyl)-3-((pyridin-4-yl)methyl)quinolin-4(1H)-one;
6,8-Dibromo-2-(4-Chloro phenyl)-3-((pyridin-4-yl)methyl)quinolin-4(1H)-one;

a stereoisomer, a derivative and/or pharmaceutically acceptable salts thereof.

The present invention also relates to process for the preparation of compound of formula (I):

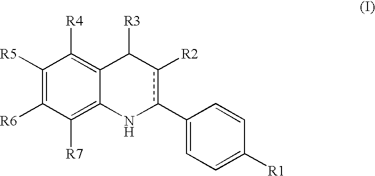

wherein, dotted line represents optional double bond;
R1 is —H, halo, —(C₁-C₃)alkyl, —O(C₁-C₃)alkyl or —CN;
R2 is —H or —(4-Pyridyl methyl);
R3 comprises -o-, heterocycle moiety, preferably of ring size 5-6; wherein the ring is selected from imidazole, triazole, tetrazole and/or pyridine; wherein further the said ring comprises substituents selected from —H, —(C₁-C₃)alkyl, —O(C₁-C₃)alkyl, F, —CF₃, —NH₂, —N(CH₃), —N(CH₃)₂, —SH, —SCH₃, —SCH₂CH₃ and/or combinations thereof;
R4, R5, R6, R7 are, independently a moiety selected from the group consisting of —H, halo, —(C₁-C₃)alkyl, —O(C₁-C₃)alkyl or —CF₃;
or a pharmaceutically acceptable salt or derivative thereof;
such as hereinafter described in SCHEME 1, comprising the steps of:
(a) synthesising 2′-Amino chalcones by condensing 2′-Amino acetophenone and para substituted benzaldehydes;
(b) cyclisation of 2′-Amino chalcones obtained in step (a) in presence of acid catalyst to prepare 2-Aryl-1,2,3,4-tetrahydro-4-quinolones;
(c) reduction of 2-Aryl-1,2,3,4-tetrahydro-4-quinolones of step (b) with NaBH₄ to give respective carbinols.
(d) further converting the respective carbinols to imidazole or triazole compounds by reacting the said carbinols with imidazole or triazole containing compounds.

The present invention also relates to process for the preparation of compound of formula (I):

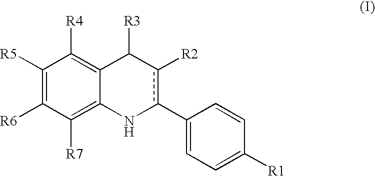

wherein, dotted line represents optional double bond;
R1 is —H, halo, —(C₁-C₃)alkyl, —O(C₁-C₃)alkyl or —CN;
R2 is —H or —(4-Pyridyl methyl);
R3 comprises -o-, heterocycle moiety, preferably of ring size 5-6; wherein the ring is selected from imidazole, triazole, tetrazole and/or pyridine; wherein further the said ring comprises substituents selected from —H, —(C₁-C₃)alkyl, —O(C₁-C₃)alkyl, F, —CF₃, —NH₂, —N(CH₃), —N(CH₃)₂, —SH, —SCH₃, —SCH₂CH₃ and/or combinations thereof;
R4, R5, R6, R7 are, independently a moiety selected from the group consisting of —H, halo, —(C₁-C₃)alkyl, —O(C₁-C₃)alkyl or —CF₃;
or a pharmaceutically acceptable salt or derivative thereof;
such as hereinbefore described in SCHEME 2, comprising the steps of:
(a) synthesising 2′-Amino chalcones by condensing 2′-Amino acetophenone and para substituted benzaldehydes;
(b) cyclisation of 2′-Amino chalcones obtained in step (a) in presence of acid catalyst to prepare 2-Aryl-1,2,3,4-tetrahydro-4-quinolones;
(c) condensation of 2-Aryl-1,2,3,4-tetrahydro-4-quinolones of step (b) with Pyridine-4-Carbaxaldehyde.

The present invention also relates to pharmaceutical composition comprising a compound of formula (I) and a pharmaceutically acceptable carrier, diluent, excipent or solvate.

In a preferred embodiment, the present invention relates to a compound of formula (I), used in the manufacture of medicament for treating or preventing cancer, particularly breast cancer, more particularly hormone dependent breast cancer.

### DETAILED DESCRIPTION OF THE INVENTION

The present invention provides novel tetrahydroquinolines compounds for the inhibition of aromatase enzyme to treat or prevent diseases associated with it, particularly breast cancer in a subject by administering therapeutically effective amount of a compound of the formula (I):

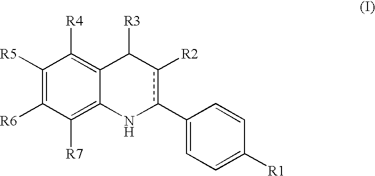

wherein,
dotted line represents optional double bond;
R1 is —H, halo, —(C₁-C₃)alkyl, —O(C₁-C₃)alkyl or —CN; R2 is —H or

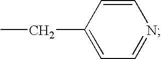

R3 comprises —O—, heterocycle moiety, preferably of ring size 5-6. The ring is selected from imidazole, triazole, tetrazole and pyridine. These rings bear substituents selected from —H, —(C₁-C₃)alkyl, —O(C₁-C₃)alkyl, F, —CF₃, —NH₂, —N(CH₃), —N(CH₃)₂, —SH, —SCH₃, —SCH₂CH₃ and combinations thereof.
R4, R5, R6, R7 are, independently a moiety selected from the group consisting of —H, halo, —(C₁-C₃)alkyl, —O(C₁-C₃)alkyl, —CF₃;
or a pharmaceutically acceptable salt or derivative thereof.

Present invention also provides for a method of using the tetrahydroquinolines compounds in the treatment of cancer. In one embodiment the novel tetrahydroquinolines compounds are used in the treatment of breast cancer, particularly but not limited to hormone dependent breast cancer. The method includes administering a therapeutically effective amount of compounds of present invention, or a derivative or pharmaceutical salt thereof, to subject in need of treatment. The compounds of the invention can be used along with other standard drugs in combination therapy for better efficacy. Also provided is a method for the prevention of breast cancer in subjects, who are susceptible to developing breast cancer, comprising administering a therapeutically effective amount of compounds of present invention, or a derivative or pharmaceutical salt there of:

Considerable data implicate steroid hormones, particularly estrogens in breast cancer carcinogenesis and progression. The mechanisms behind these phenomena are not yet fully understood but appear to be related to exposure to estrogens and its metabolites. Two thirds of postmenopausal breast cancers are positive for estrogen and progesterone receptors, and are dependent on these hormones for their proliferation. Rapid proliferation may result in genetic errors and consequent predisposition to cellular malignant potential, with morbidity and mortality. Pharmacological means of estrogen inhibition remains attractive. Until recently tamoxifen, a competitive inhibitor of the ER has been the gold standard for prevention and adjuvant and first line metastatic therapy for hormone responsive breast cancer. Recently aromatase inhibitors have challenged tamoxifen's dominance in the breast cancer therapy arena and have superseded tamoxifen in both the adjuvant and first line metastatic settings for hormone responsive beast cancer. Aromatase inhibitors are subdivided in two categories, steroidal and nonsteroidal. In contrast to steroidal aromatase inhibitors, which irreversibly inactivate the aromatase enzyme complex, nonsteroidal aromatase inhibitors reversibly interact with the heme moiety of the aromatase by completing the natural substrate androstenedione and testosterone.

The present invention discloses novel nonsteroidal aromatase inhibitors in the form of tetrahydroquinolines, methods for preparation of these novel compounds and methods of using them. The present invention encompasses quinolines, as well as salts, esters and derivatives and related compounds. The tetrahydroquinolines have the following general formula (I):

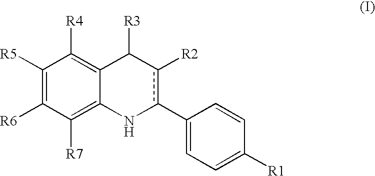

wherein,
dotted line represents optional double bond;
R1 is —H, halo, —(C₁-C₃)alkyl, —O(C₁-C₃)alkyl or —CN; R2 is —H or

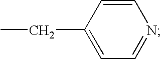

R3 comprises —O—, heterocycle moiety, preferably of ring size 5-6. The ring is selected from imidazole, triazole, tetrazole and pyridine. These rings bear substituents are selected from —H, —(C₁-C₃)alkyl, —O(C₁-C₃)alkyl, F, —CF₃, —NH₂, —N(CH₃), —N(CH₃)₂, —SH, —SCH₃, —SCH₂CH₃ and combinations thereof.
R4, R5, R6, R7 are, independently a moiety selected from the group consisting of —H, halo, —(C₁-C₃)alkyl, —O(C₁-C₃)alkyl, —CF₃;
or a pharmaceutically acceptable salt or derivative thereof.

In a preferred embodiment the tetrahydroquinolines compounds of the present invention have the general formula (I), wherein the substituents are following:

R1 is preferably from —H, Cl, Br, F, —CH₃, —OCH₃ or —CN;
R3 is preferably

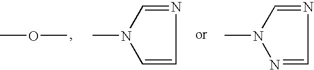

R4, R5, R6, R7 are preferably selected from H or Br.

General terms used in the description of compounds herein described bear their usual meanings. For example:
“C₁-C₃ alkyl” refers to straight or branched aliphatic chains of 1-3 carbon atoms and includes moieties such as methyl, ethyl, propyl, isopropyl and the like.
“C₁-C₃ alkoxy” refers to C₁-C₃ alkyl group attached through an oxygen molecule and include moieties such as for example, methoxy, ethoxy, n-propoxy, isopropoxy, and the like.
The term “halo” refers to bromo, chloro, fluoro, and iodo.
The term “aryl”, used alone or in combination with other terms such as alkylaryl, haloaryl or haloalkylaryl includes such aromatic rings as phenyl, biphenyl and benzyl, as well as fused aryl radicals such as napthyl, anthryl, phenanthryl, fluorenyl and indenyl on so forth.
The term “heterocycle” encompasses the aryls that have one or more heteroatoms. Such as O, N or S in the aromatic ring. Examples of heterocycles include imidazole, triazole, tetrazole, indole, pyrrole and so on.
The term “stereoisomer” refers to a compound made up of the same atoms bonded by the same bonds but having different three-dimensional structures, which are not interchangeable. The stable three-dimensional structures are called configurations.
As used herein, the term “enantiomer” refers to two stereoisomers whose molecules are non superimposable mirror images of one another.
The term “chiral center” refers to a carbon atom to which four different groups are attached.
The term “diastereoisomer” refers to stereoisomers that are not enantiomers.
Two diastereoisomers, which have different configuration at only one chiral center, are referred to herein as “epimers”.
The term “recemate”, “recemic mixture” or “recemic modification” refers to a mixture of equal parts of enantiomers.
In addition, “geometrical isomer” related to compounds containing double bond and the four atoms directly attach to them are all in the plane and that rotation around the double bond is prevented. The two groups attached to each carbon atom are ranked by Cahn-Ingold-Prelog system; the “Z isomer” refers to that isomer with the two higher ranking groups on the same side of the double bond. The “E isomer” refers to that isomer with the two higher ranking groups on the opposite side of the double bond.

The person skilled in the art will recognise that stereocentres exist in compounds of formula (I). Accordingly the present invention includes all possible stereoisomers (optical and geometric isomers) of formula (I). It also includes not only racemic compounds, or racemic mixtures thereof, but also the optically active isomers as well. When a compound of formula (I) is desired as a single enantiomer, it may be obtained either by resolution of the final product or by a stereospecific synthesis from either optically pure starting material or any convenient intermediate.

These terms and methods for identifying and selecting the desired compounds are well known in the art for example, diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from the appropriate stereochemically pure starting materials, or by using stereoselective reactions.

The compounds of present invention form pharmaceutically acceptable acid or base addition salts with a wide variety of organic and inorganic acids and bases, and include the physiologically acceptable salts which are often used in pharmaceutical chemistry such salts are also part of this invention. Typical inorganic acids used to form such salts include hydrochloric, hydrobromic, hydroiodic, nitric, sulfuric, phosphoric, hypophosphoric, and the like. Salts derived from organic acids aliphatic mono and dicarboxylic acid, phenyl substituted alkanoic acids, hydroxyalkanoic and hydroxyalkandioic acids, aromatic acids, aliphatic and aromatic sulfonic acids may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, napthelene-2-benzoate, isobutyrate, phenylbutyrate, b-hydroxybutyrate, butyne-1-4-dioate, hexyne-1-4-dioate, caprate, caprylate, cinnamate, citrate, formate, fumerate, glycollate, heptanoate, hippurate, lactate, maleate, malate, hydroxymaleate, malonate, madelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, ethanesulfonate, naphthalene-1-sulfonate, naphthalene-2-sulfonate, p-toluenesulfonate, xylenesulfonate, tartarate, and the like. Preferred salts are the hydrochloride, hydrobromide, citrate and oxalate.

Typical bases used to form pharmaceutically acceptable addition salts would be inorganic bases such as, sodium, potassium, lithium, calcium, aluminium, ammonium, barium, zinc, magnesium and the like. Further, organic bases may be utilized to form salts for example N-N′-dibenzylethelynediamine, choline, diethanolamine, ethylenediamine, N-methylglucamine, triethylamine, dimethylamine, procaine salts and the like. Also included are salts of aminoacids such as arginate and the like.

The pharmaceutically acceptable acid or base salts are typically formed by reacting a compound of formula (I) with an equimolar or excess amount of acid or base in a neat or are in a suitable inert solvent. The formed salts are further processed and purified by known methods.

The compounds of the present invention include the compounds that are in a prodrug form. A prodrug is in most cases a pharmacologically inactive derivative of a parent drug molecule that requires spontaneous or enzymatic transformation within the body in order to release the active drug, and that has improved pharmacokinetic properties over the parent drug molecule. The prodrug of a compound of general formula (I) would be a compound, which has chemically, or metabolically cleavable groups and which readily undergoes chemical changes under physiological conditions to provide a compound of formula (I) in vivo. Prodrugs include conjugation of compounds of formula (I) with sugar moieties adding suitable spacers, alkyl esters prepared by the reaction of the parent acidic compound with a suitable alcohol, or amides prepared by reaction of the parent acid compound with suitable amine so on.

“Treating” means curing, ameliorating or tampering the severity of the cancer or the symptoms or the effects associated there with. The terms “treating”, “treatment” and “therapy” as used herein refer to curative therapy, prophylactic therapy, and preventive therapy.

“Preventing” or “prevention” means preventing the occurrence of cancer or tampering the severity of cancer if it develops subsequent to the administration of the compositions. This “preventing” the onset of a clinically evident unwanted cell proliferation altogether or “preventing” the onset of a preclinical evident stage of unwanted rapid cell proliferation is in individuals/subjects at risk. This definition of prevention also encompasses the prevention of metastasis of malignant cells or to arrest or reverse the progression of malignant cells; and includes prophylactic treatment of those at risk of developing pre-cancer and cancers.

The terms “therapeutically effective” and “pharmacologically effective” are used to qualify the amount of each agent, which when used achieves the objective of improvement in disease severity and frequency of incidence over treatment of each agent by itself, while avoiding adverse side effects associated with alternative therapies.

The term “subject” for purposes of treatment includes any human or animal subject having a neoplasia such as cancer or precancer. For methods of prevention the subject is any human or animal subject, and preferably is a human subject at risk of developing a cancer. The subject may be at risk due to exposure to carcinogenic agents, being genetically predisposed to disorders characterized by unwanted, rapid cell proliferation and so on. Besides being useful for human treatment, the compounds of the present invention are useful for veterinary treatment of mammals, including companion animals and farm animals, such as, but not limited to dogs, cats, horses, cows, sheep, and pigs. Preferably, subject means a human.

The routes of administration for the compounds of formula (I) are oral, subcutaneous, intramuscular or intravenous or by any route which delivers the therapeutically effective amount of active agent to the organ or tissue or site to be treated. It will be appreciated that different dosages may be required for treating different hormone dependent cancers. Additionally the compounds of present invention may be delivered to the target site or tumor by novel techniques known to the person skilled in the art like antibody conjugation, pH sensitive polymer implants and so on. The preferred route of administration is oral.

“Pharmaceutically effective dose” means an amount of a pharmaceutical compound or composition having a therapeutically relevant effect in the frame of treatment and/or prevention of disease conditions. The dosage also depends upon a variety of factors, including age, weight, sex, medical condition of the patient, the severity of disease, the route and frequency of administration, potency of the compound employed, the site of the proliferation, as well as pharmacokinetic properties of the compound being administered, adverse and toxicological effects of the compound being used and interaction of the present invention compound with other agents. Generally the dosage of compounds administered locally rather than systemically and for prevention rather than treatment will be lower. Such treatments are administered as often as necessary and for the period of time judged necessary by the treating physician and veterinarian. The dosage regime or therapeutically effective amount of the compound to be administered may need to be optimized for each subject. A typical daily dose for the compounds contains nontoxic dosage level from about 1 mg to about 800 mg/day of a compound. Preferred daily doses generally are from about 1 mg to 200 mg/day. Most preferred doses range constitute 1 mg, 2 mg, 5 mg, 10 mg, 20 mg, 30 mg, 40 mg, 50 mg, 60 mg, 70 mg, 90 mg and 100 mg. The compounds or compositions (including combination with additional agent) of the present invention may be administered in a single dose, or the total daily dosage may be administered in individual doses divided into two, three, or four times daily. Similarly, the treatment can be adapted to administer the compounds or compositions (including combinations) of the invention in a single weekly or monthly dose.

According to present invention, the expression “used in combination with” means that the administration of compounds of formula (I) simultaneously or consecutively with one or more other pharmaceutical agent, simultaneously refers to co-administration. In this case, the separate components of the combination can be mixed to form a single composition prior to being administered, or can be administered at the same time to the patient. It is also possible to administer them consecutively that is to say one after the other, irrespective of which component of the combination according to the invention is administered first. Using a mode of administration, which is staggered over time or is intermittent and which stops and restarts at intervals which may or may not be regular is within the scope of this invention. It is pointed out that the routes and sites of administration of the two components can be different. The time interval between the administrations is not critical and can be defined by the skilled person.

Examples for additional agents of combination therapy are other large numbers of antineoplastic agents available in market, in clinical trials, or in preclinical evaluation, which could be selected for treatment of cancers or other neoplasias by combination drug chemotherapy. These agents fall into several major categories, namely alkylating agents, antimetabolites, antibiotics, immunomodulating agents, nucleotide derivatives, cyclin dependent kinase inhibitors, interferon like agents and histone deacytalase inhibitors and so on.

Other preferred agents for combination therapy are COX-II inhibitors, which serve as selective aromatase expression regulators examples for such category of compounds include nimuselide, celocoxib, etorocoxib, valdicoxib and like.

Accordingly, the present invention concerns with a composition comprising at least one compound of the present invention and pharmaceutically acceptable carrier or diluent thereof. These pharmaceutical compositions are prepared by conventional techniques. Typical compositions of the present invention are associated with pharmaceutically acceptable excipients which may be a carrier or a diluent or be diluted by a carrier, or enclosed with in a carrier which can be in the form of a capsule, sachet, tablets, aerosols, solutions, suspensions, injectables or other compositions. In making combination products, conventional techniques for the preparation of pharmaceutical composition may be used. For example, the active compound is usually be mixed with a carrier or a diluent, or diluted by carrier or a diluent, or enclosed within a carrier or diluent which may be in the form of a injectable, capsule, sachet, tablets, aerosols, solutions, suspensions or other compositions. When the other carrier serves as a diluent, it may be solid, semisolid, or liquid material, which acts as a vehicle, excipient, or medium for the active compound.

Some examples of suitable carriers or diluents are without being limited, water, salt solutions, alcohols, polyethylene glycols, polyhydroxy ethoxylated castor oil, peanut oil, olive oil, gelatine, lactose, sucrose, cyclodextrin, amylose, magnesium stereate, talc, agar, silicic acid, fatty acids, fatty acid amines, fatty acid monoglycerides, fatty acid diglycerides, polyoxyethylene, hydroxymethylcellulose, and polyvinylpyrrolidine.

Tablets and capsules represent the most advantageous oral dosage unit form, in which case solid pharmaceutical carriers are obviously employed. The compounds also can be formulated as elixirs or solutions for convenient oral administration. The active compounds are formulated as appropriate solutions for parenteral administration for example by intravenous, intramuscular, and subcutaneous. Additionally the carrier or diluent may include any sustain release material known in the art, such as glyceryl monostereate or glyceryl distereate. In one embodiment the active compounds are incorporated into controlled release formulation, including implants and microencapsulated delivery systems. The compounds of the present invention can also be administered in the liposomes or nanoparticle delivery systems. The composition can be so constituted that they release the active ingredient only or preferably in a particular physiological location, possibly over a period of time.

More specifically, the invention concerns compounds contemplated as falling within the scope of the formula (I). Compounds include, but are not limited to the following compounds and their pharmaceutically acceptable salts:
2-Phenyl-4-imidazol-1-yl-1,2,3,4-tetrahydro-quinoline;
2-(p-Tolyl)-4-imidazol-1-yl-1,2,3,4-tetrahydro-quinoline;
2-(4-Methoxy phenyl)-4-imidazol-1-yl-1,2,3,4-tetrahydro-quinoline;
2-(4-Chloro phenyl)-4-imidazol-1-yl-1,2,3,4-tetrahydro-quinoline;
2-(4-Bromo phenyl)-4-imidazol-1-yl-1,2,3,4-tetrahydro-quinoline;
4-(4-Imidazol-1-yl-1,2,3,4-tetrahydro-quinolin-2-yl)-benzonitrile;
2-(4-Fluoro phenyl)-4-imidazol-1-yl-1,2,3,4-tetrahydro-quinoline;
2-Phenyl-4-[1,2,4]triazol-1-yl-1,2,3,4-tetrahydro-quinoline;
2-(p-Tolyl)-4-[1,2,4]triazol-1-yl-1,2,3,4-tetrahydro-quinoline;
2-(4-Methoxy phenyl)-4-[1,2,4]triazol-1-yl-1,2,3,4-tetrahydro-quinoline;
2-(4-Chloro phenyl)-4-[1,2,4]triazol-1-yl-1,2,3,4-tetrahydro-quinoline;
2-(4-Bromo phenyl)-4-[1,2,4]triazol-1-yl-1,2,3,4-tetrahydro-quinoline;
4-(4-[1,2,4]Triazol-1-yl-1,2,3,4-tetrahydro-quinolin-2-yl)-benzonitrile;
2-(4-Fluoro phenyl)-4-[1,2,4]triazol-1-yl-1,2,3,4-tetrahydro-quinoline;
2-Phenyl-3-((pyridin-4-yl)methyl)quinolin-4 (1H)-one;
2-(p-Tolyl)-3-((pyridin-4-yl)methyl)quinolin-4(1H)-one;
2-(4-Methoxy phenyl)-3-((pyridin-4-yl)methyl)quinolin-4(1H)-one;
2-(4-Chloro phenyl)-3-((pyridin-4-yl)methyl)quinolin-4(1H)-one;
2-(4-Bromo phenyl)-3-((pyridin-4-yl)methyl)quinolin-4(1H)-one;
4-(4-oxo-3-((pyridin-4-yl)methylene)quinolin-2-yl)benzonitrile;
2-(4-Fluoro phenyl)-3-((pyridin-4-yl)methyl)quinolin-4(1H)-one;
6,8-Dibromo-2-phenyl-3-((pyridin-4-yl)methyl)quinolin-4(1H)-one;
6,8-Dibromo-2-(p-tolyl)-3-((pyridin-4-yl)methyl)quinolin-4(1H)-one;
6,8-Dibromo-2-(4-Methoxy phenyl)-3-((pyridin-4-yl)methyl)quinolin-4 (1H)-one;
6,8-Dibromo-2-(4-Chloro phenyl)-3-((pyridin-4-yl)methyl)quinolin-4 (1H)-one;
   The synthesis of target compounds is carried by the process outlined in scheme 1 and scheme 2.

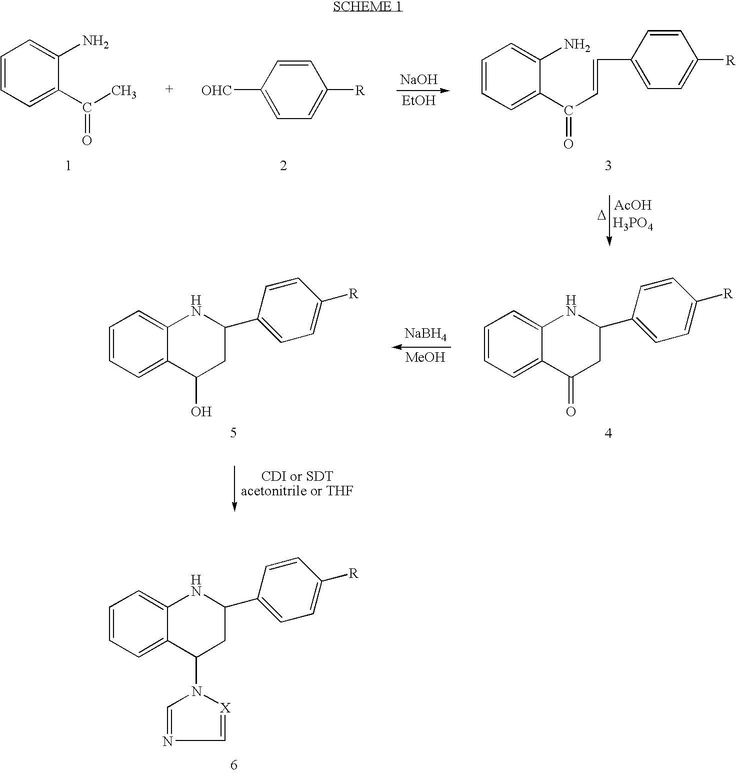

The following compounds are synthesized from scheme I:

| Compound code | R | X |
|---|---|---|
| 6a | —H | C |
| 6b | —CH₃ | C |
| 6c | —OCH₃ | C |
| 6d | —Cl | C |
| 6e | —Br | C |
| 6f | —CN | C |
| 6g | —F | C |
| 6h | —H | N |
| 6i | —CH₃ | N |
| 6j | —OCH₃ | N |
| 6k | —Cl | N |
| 6l | —Br | N |
| 6m | —CN | N |
| 6n | —F | N |

The following protocol was adopted for preparing compounds from scheme 1:
In first step, 2′-amino acetophenone (1) (10 mmol) is condensed with para substituted benzaldehyde (2) (10 mmol) in ethanol (10 mL) medium containing catalytic amount of NaOH (˜200 mg, 2 pellets), then stirring the mixture at room temperature or at 0-5° C. for 3 to 12 hrs. The resultant precipitate is filtered and washed with cold distilled water, recrystalised by suitable solvent (Wattinasian, S. and Murphy, W. S., Synthesis, 1980, 647-650; Li, R et al J. Med. Chem., 1995, 38, 5031-5037).
In second step, chalcones (3) are cyclised by acid catalysis.
   In one method a mixture of 2′-aminochalcone (3 mmol), glacial acetic acid (12.5 mL) and orthophosphoric acid (12.5 mL) is warmed at 100° C. for ½ to 2 hr. After cooling, the mixture is poured into iced water. The product hence precipitated is purified by column chromatography on silica gel using hexane:ethylacetate 85:15 v/v (Tokes, A. L., Litkei, Gy., Szilagyi, L., Syn. Comm, 1992, 22, 2433-2445).
   Other method applied for chalcone cyclisation is microwave-assisted isomerisation. The procedure followed is montmorillonite K 10 clay (1.0 g) is mixed with 2′-aminochalcone (100 mg) in dichloromethane (2 mL). The adsorbed material is transferred to a test tube and is inserted in an alumina bath (alumina; mesh 65-325; bath of 5.7 cm diameter) inside the microwave oven. The compound is irradiated for 1.5 min (the temperature of alumina bath reaches 110° C. at the end of this period). The product is extracted into dichloromethane and clay is filtered off (Verma, R. S and Saini, R. K, Syn. Lett, 1997, 857-858).

The former method is preferred over the latter due to purity and yield advantages.
In third step of scheme 1, the 2-aryl-1,2,3,4-tetrahydro-4-quinolones are reduced to respective carbinols by NaBH₄ reduction. The procedure followed is to a suspension of 2-aryl-1,2,3,4-tetrahydro-4-quinolones (1 mmol) in dry methanol (10 mL) is added to NaBH₄ (4 mmol) and the reaction mixture is stirred at 0-5° C. under nitrogen atmosphere for 3 hrs. The reaction mixture is concentrated under reduced pressure and 2M aqueous hydrochloric acid (˜2 mL) is added to resulting solution to adjust pH of the solution to 6. This solution is extracted into diethyl ether (20 mL), washed with water (2×15 mL), dried over NaSO₄ and concentrated under reduced pressure to give required product in quantitative yield (Pouget, C et al., Bioorg. Chem., 2004, 32, 494-503; Chaikin, S. W and Brown, W G., J. Am. Chem. Soc, 1949, 71, 122-125).
Finally, imidazole containing compounds are synthesized from carbinols by stirring the corresponding carbinol (1 mmol) and N,N′-carbonyl diimidazole (4 mmol) in dry tetrahydrofuran (20 mL) under nitrogen atmosphere at room temperature for overnight. The reaction mixture is evaporated to dryness. The residue is dissolved in chloroform, organic solution is washed with water, dried over Na₂SO₄. The crude product is purified by column chromatography on alumina using chloroform:acetone 90:10 v/v (Njar, V. C. O., Synthesis, 2000, 2019-2028; Leze, M. P et al., J. Enz. Inh. Med. Chem., 2004, 1-9).
   Triazole containing compounds are obtained by adopting the procedure as follows: (a) to a cooled (0-5° C.) solution of 1,2,4,-triazole (4 mmol) in dry acetonitrile (10 mL) is added a solution of thionyl chloride (1 mmol) in dry acetonitrile (1 mL); (b) the reaction is stirred at (0-5° C.) for 1 hr. The solution prepared by this procedure is filtered and added dropwise to solution of the carbinols (1 mmol) in dry acetonitrile (10 mL).
   The reaction mixture is stirred under nitrogen atmosphere at room temperature for overnight then it is filtered to remove solid residues and then concentrated. The residue is dissolved in chloroform. The organic solution is washed with brine, dried over Na₂SO₄, crude product is purified by column chromatography on alumina using hexane:ethyl acetate 75:25 v/v (Saberi, M. R. et al., J. Med. Chem., 2006, 49, 1016-1022; Massa, S et al., Eur. J. Med. Chem., 1992, 27, 495-502).

If in any of the processes mentioned herein, the substituting moiety is different from the one required, the substituting moiety may be converted to the desired moiety by known methods. The substituting moiety may also need protection against the conditions under which reactions are carried out, accordingly, a protecting group may be used which is removed after reactions have been completed.

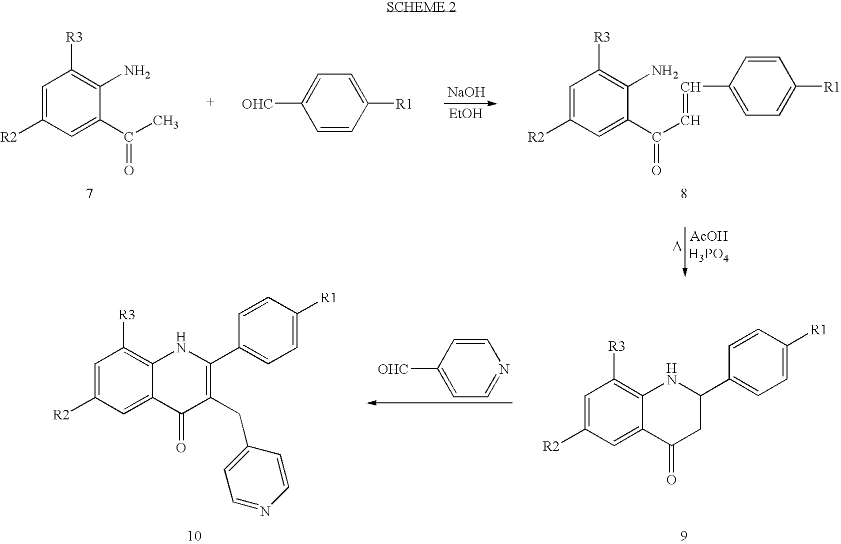

The following compounds are synthesized from scheme 2:

| Compound code | R1 | R2 | R3 |
|---|---|---|---|
| 10a | —H | —H | —H |
| 10b | —CH₃ | —H | —H |
| 10c | —OCH₃ | —H | —H |
| 10d | —Cl | —H | —H |
| 10e | —Br | —H | —H |
| 10f | —CN | —H | —H |
| 10g | —F | —H | —H |
| 10h | —H | —Br | —Br |
| 10i | —CH₃ | —Br | —Br |
| 10j | —OCH₃ | —Br | —Br |
| 10k | —Cl | —Br | —Br |

The following protocol was adopted for preparing compounds from scheme 2:
In first step, 2′-amino acetophenones (7) 10 mmol) is condensed with para substituted benzaldehyde (2) (10 mmol) in ethanol (10 mL) medium containing catalytic amount of NaOH (˜200 mg, 2 pellets), by stirring the mixture at room temperature or at 0-5° C. for 3 to 12 hrs. The resultant precipitate is filtered and washed with cold distilled water, recrystalised by suitable solvent (Wattinasian, S and Murphy, W. S., Synthesis, 1980, 647-650; Li, R et al., J. Med. Chem., 1995, 38, 5031-5037).
In second step, chalcones (8) are cyclised by acid catalysis.

In one method a mixture of 2′-aminochalcone (3 mmol), glacial acetic acid (12.5 mL) and orthophosphoric acid (12.5 mL) is warmed at 100° C. for ½ to 2 hr. After cooling the mixture is poured into iced water. The product precipitated is purified by column chromatography on silicagel using hexane:ethylacetate 85:15 v/v (Tokes, A. L., Litkei, Gy., Szilagyi, L., Syn. Comm, 1992, 22, 2433-2445).

Other method applied for chalcone cyclisation is microwave-assisted isomerisation. The procedure followed is montmorillonite K 10 clay (1.0 g) is mixed with 2′-aminochalcone (100 mg) in dichloromethane (2 mL). The adsorbed material is transferred to a test tube and is inserted in an alumina bath (alumina; mesh 65-325; bath of 5.7 cm diameter) inside the microwave oven. The compound is irradiated for 1.5 min (the temperature of alumina bath reaches 110° C. at the end of this period). The product is extracted into dichloromethane and clay is filtered off (Verma, R. S and Saini, R. K., Syn. Lett, 1997, 857-858).

The preferred method is former over latter due to purity advantages.
In third step, 2-aryl-1,2,3,4-tetrahydro 4 quinolones (9) are condensed with pyridine-4-carboxaldehyde. Two methods are followed for the condensation.
In one method, equivalent amounts of 2-aryl-1,2,3,4-tetrahydro I quinolones (9) and pyridine-4-carboxaldehyde are allowed to react at 130° C. in the presence of few drops of piperidine for 2 hr. Then chloroform is added to the reaction mixture and the organic layer is washed twice with water, the residue is purified by column chromatography on silica gel with hexane:ethyl acetate 60:40 v/v (Pouget, C et al., Bioorg. Med. Chem., Lett, 2002, 12, 1059-1061).
The other method is, aqueous sodium hydroxide solution (6N, 2 mL) is added to a solution of 2-aryl-1,2,3,4-tetrahydro 4 quinolones (1 mmol) in 5 mL of ethanol. Pyridine-4-carboxaldehyde (1.2 mmol) is added to reaction mixture, which is stirred at room temperature for 24 hr. Ethanol is evaporated under reduced pressure, precipitate is dissolved in chloroform and washed with water. The crude product is purified by column chromatography on silica gel using ethylacetate:acetone 97:3 v/v (Auvray. P et al., Bioorg. Med. Chem., 2000, 8, 945-955).
Latter method is preferred over former because of high yield and ease of work up advantageous.
   If in any of the processes mentioned herein, the substituting moiety is different from the one required, the substituting moiety may be converted to the desired moiety by known methods. The substituting moiety may also need protection against the conditions under which reactions are carried out, accordingly, a protecting group may be used which is removed after reactions have been completed.

### EXAMPLES

Unless otherwise stated, chemicals used herein are commercially available and used as received without further purification. Dry nitrogen atmosphere was used for moisture sensitive reactions. Dry methanol from Merck; tetrahydrofuran was distilled from sodium metal with benzophenone; acetonitrile was dried by distilling from molecular sieve 4° A and calcium hydride. All heating reactions were carried out on 12-place reaction station of Radleys discovery Technologies. Thin layer chromatography was performed on precoated silica gel F₂₅₄ (Merck). Column chromatography was performed using silica gel 60-120 mesh and alumina activity I-II grade (Merck). Melting points, were determined in open glass capillaries using a Polmon melting point apparatus and uncorrected. Infra red spectra recorded on Perkin-Elmer infrared spectrophotometer in KBR pellet. Mass spectra obtained on VG-7070H mass spectrometer. NMR spectra were recorded at 300 MHz on a Bruker avance NMR spectrometer in CDCL₃ (δ 7.26) or DMSO-d₆ (δ 2.49).

The novel compounds of the present invention were prepared according to the following procedures, using appropriate materials and are further exemplified by the following specific examples. The most preferred compounds of the invention are any or all of those specifically set forth in these examples. These compounds are not, however, to be construed as forming the only genus that is considered as the invention, and any combination of the compounds or their moieties may itself form a genus. The following examples further illustrate details for the preparation of the compounds of the present invention. Those skilled in the art will readily understand that known variations of the conditions and process of the following preparative procedures can be used to prepare these compounds.

### Example 1

### Preparation of 1-(2-Amino-phenyl)-3-propenone of Formula (II)

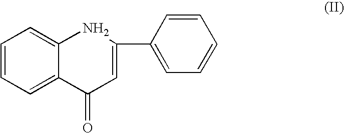

2′-Amino acetophenone (1.35 g, 10 mmol) is added to solution of benzaldehyde (1.06 g, 10 mmol) in 10 mL of ethanol containing NaOH (200 mg, 2 pellets), stirred at 0-5° C. for 12 hr. The precipitate is filtered, washed with cold water and recrystallized from ethanol. Yield 1.5 g (62%); mp 70-72° C.; IR (KBR) 3316, 1647, 1209 cm⁻¹; MS m/z 224 (M+H)⁺.

### Example 2

### Preparation of 4-[3-(2-Amino-phenyl)-3-oxo-propenyl]benzonitrile of Formula (III)

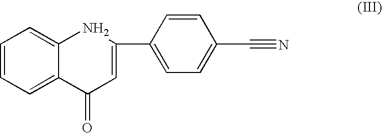

2′-Amino acetophenone (1.35 g, 10 mmol) is added to solution of p-cyano benzaldehyde (1.31 g, 10 mmol) in 10 mL of ethanol containing NaOH (200 mg, 2 pallets), stirred at room temperature for 4 hr. The precipitate is filtered, washed with cold water and recrystallized from methanol. Yield 1.8 g (72%); mp 126-128° C.; ¹H NMR (CDCL₃) δ: 6.76 (m, 2H, H-3′ and H-5′), 7.24-7.92 (m, 8H, H-2, H-3, H-5, H-6, H-4′, H-6′, H-α and H-β).

### Example 3

### Preparation of 2-p-Tolyl-1,2,3,4-tetrahydro-4-quinolone of Formula (IV)

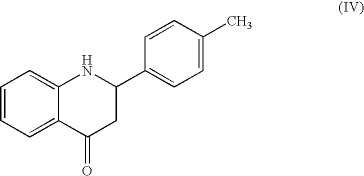

Mixture of 1-(2-amino-phenyl)-3-p-tolyl-propenone (710 mg, 3 mmol), glacial acetic acid (12.5 mL) and orthophosphoric acid (12.5 mL) is warmed at 100° C. for 40 min. After cooling the mixture is poured into iced water. The product precipitated is purified by column chromatography on silica gel using hexane:ethylacetate 85:15 v/v. Yield 400 mg (56%); mp 144-146° C.; IR (KBR) 3313, 1650 cm⁻¹; ¹H NMR (CDCL₃) δ 2.4 (s, 3H, CH₃), 2.8 (m, 2H, H-3e, H-3a), 4.5 (s, 1H, NH), 4.8 (dd, 1H, H-2), 6.7-7.4 (m, 7H, Aro), 7.9 (dd, 1H, H-5).

### Example 4

### Preparation of 4-(4-Oxo-1,2,3,4-tetrahydro-quinolin-2-yl)-benzonitrile of Formula (V)

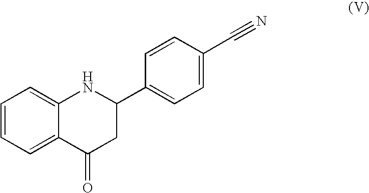

Mixture of 4-[-3-(2-amino-phenyl)-3-oxo-propenyl]benzonitrile (744 mg, 3 mmol), glacial acetic acid (12.5 mL) and orthophosphoric acid (12.5 mL) is warmed at 100° C. for 2 hr. After cooling the mixture is poured into iced water. The product precipitated is purified by column chromatography on silicagel using hexane:ethylacetate 85:15 v/v. Yield 360 mg (56%); mp 206-208° C.; ¹H NMR (CDCL₃) δ 2.8 (m, 2H, H-3e, H-3a), 4.5 (br, 1H, NH), 4.8 (dd, 1H, H-2), 7.3-7.7 (m, 7H, Aro), 7.9 (dd, 1H, H-5).

### Example 5

### Preparation of 2-Phenyl-1,2,3,4,-tetrahydro-quinolin-4-ol of Formula (VI)

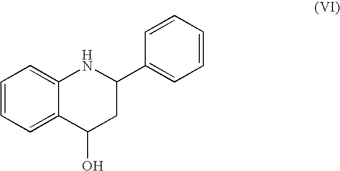

To a suspension of 2-phenyl-1,2,3,4-tetrahydro-4-quinolones (223 mg, 1 mmol) in dry methanol (10 mL) is added NaBH₄ (4 mmol) and the reaction mixture stirred at 0-5° C. under nitrogen atmosphere for 3 hr. The reaction mixture is concentrated under reduced pressure and 2M aqueous hydrochloric acid (˜2 mL) is added to adjust the pH 6. This solution is extracted into diethyl ether (20 mL), washed with water (2×15 mL), dried over NaSO₄ and concentrated under reduced pressure to give required product. Yield 210 mg (93%); IR (KBR) 3304, 1453, 1209 cm⁻¹; MS m/z 226.1 (M+H)⁺.

### Example 6

### Preparation of 2-p-Tolyl-1,2,3,4,-tetrahydro-quinolin-4-ol of Formula (VII)

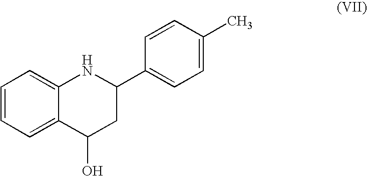

To a suspension of 2-p-tolyl-1,2,3,4-tetrahydro-4-quinolones (237 mg, 1 mmol) in dry methanol (10 mL) is added NaBH₄ (4 mmol) and the reaction mixture is stirred at 0-5° C. under nitrogen atmosphere for 3 hr. The reaction mixture is concentrated under reduced pressure and 2M aqueous hydrochloric acid (˜2 mL) is added to adjust the pH 6. This solution is extracted into diethyl ether (20 mL), washed with water (2×15 mL), dried over NaSO₄ and concentrated under reduced pressure to give required product. Yield 190 mg (78%); IR (KBR) 3250, 1482 cm⁻¹; ¹H NMR (CDCL₃) δ 2.1 (m, 1H, 3a), 2.4 (m, 41-1, H-3e, CH3), 3.9 (s, 1H, NH), 4.6 (dd, 1H, H-2), 5.1 (br, 1H, H-4) 6.5-7.5 (m, 8H, Aro).

### Example 7

### Preparation of 2-Phenyl-4-imidazol-1-yl-1,2,3,4-tetrahydro-quinoline of Formula (VIII)

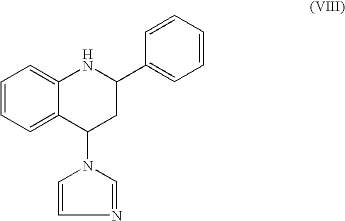

2-Phenyl-1,2,3,4,-tetrahydro-quinolin-4-ol (225 mg, 1 mmol) and N,N′-carbonyl diimidazole (640 mg, 4 mmol) in dry tetrahydrofuran (20 mL) are stirred under nitrogen atmosphere at room temperature for overnight. The reaction mixture is evaporated to dryness. The residue is dissolved in chloroform (20 mL), organic solution is washed with water (2×15 mL), and dried over Na₂SO₄. The crude product is purified by column chromatography on alumina using chloroform:acetone 90:10 v/v. Yield 85 mg (30%); mp 170-172° C. IR (KBR) 3241, 3103 cm⁻¹; ¹H NMR (CDCL₃) δ 2.32 (q, 2H, H-3a and H-3e), 4.26 (t, 2H, NH and H-2), 5.30 (t, 1H, H-4), 6.69 (q, 2H, H-8 and H-6), 6.92 (s, 1H, H-4″), 7.04 (d, 1H, H-5), 7.10 (s, H-1, H-5″), 7.19-7.35 (m, 6H, H-2′, H-3′, H-4′, H-5′, H-6′, and H-7), 7.43 (s, 1H, H-2″); MS m/z 276.3 (M+H)⁺, 208.2 (M+H-68, imidazole)⁺.

### Example 8

### Preparation of 2-(p-Tolyl)-4-imidazol-1-yl-1,2,3,4-tetrahydro-quinoline of Formula (IX)

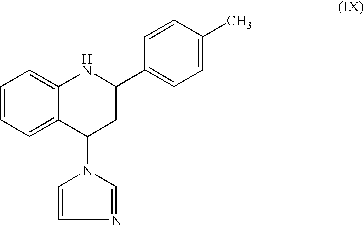

2-p-Tolyl-1,2,3,4,-tetrahydro-quinolin-4-ol (240 mg, 1 mmol) and N,N′-carbonyl diimidazole (640 mg, 4 mmol) in dry tetrahydrofuran (20 mL) are stirred under nitrogen atmosphere at room temperature for overnight. The reaction mixture is evaporated to dryness. The residue is dissolved in chloroform (20 mL), organic solution is washed with water (2×15 mL), dried over Na₂SO₄. The crude product is purified by column chromatography on alumina using chloroform:acetone 90:10 v/v. Yield 70 mg (29%); mp 152-154° C. IR (KBR) 3250, 2929 cm⁻¹; ¹H NMR (CDCL₃) δ 2.34 (m, 5H, H-3a, H-3e and CH₃), 4.23 (t, 2H, NH and H-2), 5.29 (t, 1H, H-4), 6.64 (d, 1H, H-8), 6.71 (t, 1H, H-6), 6.91 (s, 1H, H-4″), 7.00 (d, 1H, H-5), 7.09 (s, 1H, H-5″), 7.14-7.26 (m, 5H, H-2′, H-3′, H-5′, H-6′ and H-7), 7.42 (s, 1H, H-2″); MS m/z 290 (M+H)⁺, 222 (M+H-68, imidazole)⁺.

### Example 9

### Preparation of 2-(4-Methoxyphenyl)-4-imidazol-1-yl-1,2,3,4-tetrahydro-quinoline of Formula (X)

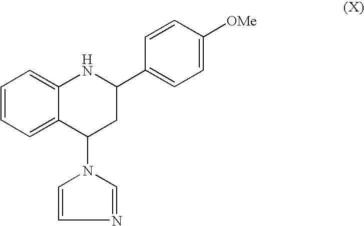

2-(4-Methoxyphenyl)-1,2,3,4,-tetrahydro-quinolin-4-ol (255 mg, 1 mmol) and N,N′-carbonyl diimidazole (640 mg, 4 mmol) in dry tetrahydrofuran (20 mL) are stirred under nitrogen atmosphere at room temperature for overnight. The reaction mixture is evaporated to dryness. The residue is dissolved in chloroform (20 mL), organic solution is washed with water (2×15 mL), dried over Na₂SO₄. The crude product is purified by column chromatography on alumina using chloroform:acetone 90:10 v/v. Yield 120 mg (46%); mp 156-158° C.; ¹H NMR (CDCL₃) δ 2.28 (q, 2H, H-3a and H-3e), 3.81 (s, 3H, OCH₃), 4.22 (q, 2H, NH and H-2), 5.28 (t, 1H, H-4), 6.66 (d, 1H, H-8), 6.71 (t, 1H, H-6), 6.84-7.46 (9H, H-5, H-7, H-2′, H-3′, H-5′, H-6′, H-2″, H-4″ and H-5″).

### Example 10

### Preparation of 2-(4-Chlorophenyl)-4-imidazol-1-yl-1,2,3,4-tetrahydro-quinoline of Formula (XI)

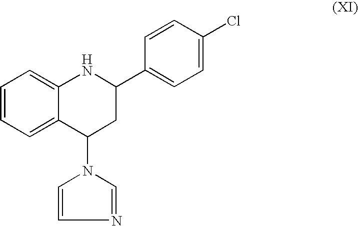

2-(4-Chlorophenyl)-1,2,3,4,-tetrahydro-quinolin-4-ol (260 mg, 1 mmol) and N,N′-carbonyl diimidazole (640 mg, 4 mmol) in dry tetrahydrofuran (20 mL) are stirred under nitrogen atmosphere at room temperature for overnight. The reaction mixture is evaporated to dryness. The residue is dissolved in chloroform (20 mL), organic solution is washed with water (2×15 mL), dried over Na₂SO₄. The crude product is purified by column chromatography on alumina using chloroform: acetone 90:10 v/v. Yield 120 mg (38%); mp 158-160° C.

### Example 11

### Preparation of 2-(4-Bromo phenyl)-4-imidazol-1-yl-1,2,3,4-tetrahydro-quinoline of Formula (XII)

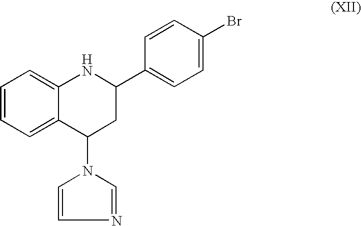

2-(4-Bromophenyl)-1,2,3,4,-tetrahydro-quinolin-4-ol (304 mg, 1 mmol) and N,N′-carbonyl diimidazole (640 mg, 4 mmol) in dry tetrahydrofuran (20 mL) are stirred under nitrogen atmosphere at room temperature for overnight. The reaction mixture is evaporated to dryness. The residue is dissolved in chloroform (20 mL), organic solution is washed with water (2×15 mL), dried over Na₂SO₄. The crude product is purified by column chromatography on alumina using chloroform: acetone 90:10 v/v. Yield 130 mg (43%); mp 142-144° C.; IR (KBR) 3243, 2933 cm⁻¹; ¹H NMR (CDCL₃) δ 2.27 (q, 2H, H-3a and H-3e), 4.25 (q, 2H, NH and H-2), 5.28 (t, 1H, H-4), 6.68 (d, 1H, H-8), 6.73 (t, 1H, H-6), 6.90 (s, 1H, H-4″), 7.01 (d, 1H, H-5), 7.09 (s, 1H, H-5″), 7.16-7.51 (m, 6H, H-2′, H-3′, H-5′, H-6′, H-7 and H-2″).

### Example 12

### Preparation of 4-(4-Imidazol-1-yl-1,2,3,4-tetrahydro-quinolin-2-yl)-benzonitrile of Formula (XIII)

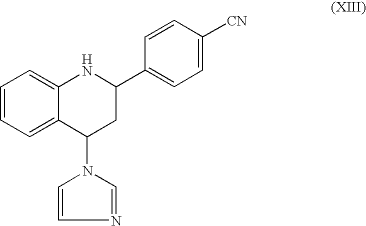

4-(4-Hydroxy-1,2,3,4-tetrahydroquinolin-2-yl)-benzonitrile (250 mg, 1 mmol) and N,N′-carbonyl diimidazole (640 mg, 4 mmol) in dry tetrahydrofuran (20 mL) are stirred under nitrogen atmosphere at room temperature for overnight. The reaction mixture is evaporated to dryness. The residue is dissolved in chloroform (20 mL), organic solution is washed with water (2×15 mL), dried over Na₂SO₄. The crude product is purified by column chromatography on alumina using chloroform:acetone 90:10 v/v. Yield 110 mg (36%); mp 166-168° C.; ¹H NMR (DMSO-d₆) δ 2.3 (m, 2H, H-3a and H-3e), 4.4 (d, 1H, H-2), 5.29 (s, 1H, H-4), 6.52-7.91 (m, 11H, H-5, H-6, H-7, H-8, H-2′, H-3′, H-5′ H-6′, H-2″, H-4″ and H-5″).

### Example 13

### Preparation of 2-(4-Fluoro phenyl)-4-imidazol-1-yl-1,2,3,4-tetrahydro-quinoline of Formula (XIV)

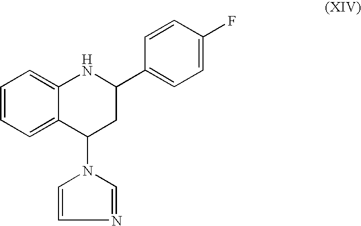

2-(4-Fluorophenyl)-1,2,3,4,-tetrahydro-quinolin-4-ol (243 mg, 1 mmol) and N,N′-carbonyl diimidazole (640 mg, 4 mmol) in dry tetrahydrofuran (20 mL) are stirred under nitrogen atmosphere at room temperature for overnight. The reaction mixture is evaporated to dryness. The residue is dissolved in chloroform (20 mL), organic solution is washed with water (2×15 mL), dried over Na₂SO₄. The crude product is purified by column chromatography on alumina using chloroform: acetone 90:10 v/v. Yield 110 mg (37%); mp 168-170° C.; ¹H NMR (CDCL₃) δ 2.28 (q, 21-1, H-3a and H-3e), 4.25 (q, 2H, NH and H-2), 5.29 (t, 1H, H-4), 6.68 (d, 1H, H-8), 6.73 (t, 1H, H-6), 6.91 (s, 1H, H-4″), 7.00-7.30 (m, 7H, H-2′, H-3′, H-5′, H-6′, H-5, H-7 and H-5″), 7.41 (s, 1H, H-2″). MS m/z 294 (M+H)⁺, 226 (M+H-68, imidazole)⁺.

### Example 14

### Preparation of 2-Phenyl-4-[1,2,4]triazol-1-yl-1,2,3,4-tetrahydro-quinoline of Formula (XV)

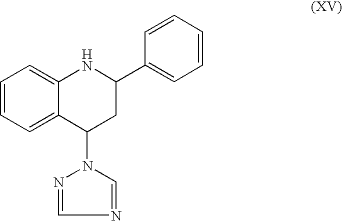

To a cooled (0-5° C.) solution of 1,2,4,-triazole (275 mg, 4 mmol) in dry acetonitrile (10 mL) is added a solution of thionyl chloride (120 mg, 1 mmol) in dry acetonitrile (1 mL) and the reaction is stirred at (0-5° C.) for 1 hr. The solution is filtered and added dropwise to solution of 2-phenyl-1,2,3,4-tetrahydro-quinolin-4-ol (225 mg, 1 mmol) in dry acetonitrile (10 mL). The reaction mixture is stirred under nitrogen atmosphere at room temperature for overnight, then it is filtered to remove solid residues and concentrated. The residue is dissolved in chloroform. The organic solution is washed with brine, dried over Na₂SO₄, crude product is purified by column chromatography on alumina using hexane:ethyl acetate 75:25 v/v. Yield 130 mg (47%); mp 162-164° C.; IR (KBR) 3280, 2924 cm⁻¹; ¹H NMR (CDCL₃) δ 2.29 (ddd, 1H, H-3e), 2.67 (d, 1H, H-3a), 4.13 (dd, 1H, H-2), 4.29 (brs, 1H, NH), 5.52 (t, 1H, H-4), 6.69 (d, 1H, H-8), 6.76 (t, 1-H, H-6), 7.12 (d, H-5), 7.20-7.38 (m, 6H, H-7, H-2′, H-3′, H-4′, H-5′ and H-6′), 7.82 (s, 1H, H-3″), 8.04 (s, 1H, H-5″); MS m/z 277 (M+H)⁺, 208 (M+H-69, Triazole)⁺.

### Example 15

### Preparation of 2-p-Tolyl-4-[1,2,4]triazol-1-yl-1,2,3,4-tetrahydro-quinoline of Formula (XVI)

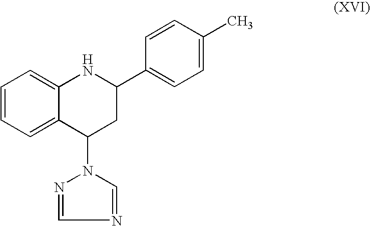

To a cooled (0-5° C.) solution of 1,2,4,-triazole (275 mg, 4 mmol) in dry acetonitrile (10 mL) is added a solution of thionyl chloride (120 mg, 1 mmol) in dry acetonitrile (1 mL) and the reaction is stirred at (0-5° C.) for 1 hr. The solution is filtered and added dropwise to solution of 2-p-tolyl-1, 2, 3,4-tetrahydro-quinolin-4-ol (240 mg, 1 mmol) in dry acetonitrile (10 mL). The reaction mixture is stirred under nitrogen atmosphere at room temperature for overnight, then it is filtered to remove solid residues and concentrated. The residue is dissolved in chloroform. The organic solution is washed with brine, dried over Na₂SO₄, crude product is purified by column chromatography on alumina using hexane:ethyl acetate 75:25 v/v. Yield 70 mg (24%); mp 146-148° C.; IR (KBR) 3354, 2922 cm⁻¹; ¹H NMR (CDCL₃) δ 2.26 (ddd, 1H, H-3e), 2.34 (s, 3H, CH₃) 2.63 (d, 1H, H-3a), 4.10 (dd, 1H, H-2), 4.26 (brs, 1H, NH), 5.51 (t, 1H, H-4), 6.67 (d, 1H, H-8), 6.74 (t, 1-H, H-6), 7.11 (d, H-5), 7.13-7.25 (m, 5H, H-7, H-2′, H-3′, H-5′ and H-6′), 7.79 (s, 1H, H-3″), 8.04 (s, 1H, H-5″); MS m/z 291 (M+H)⁺, 222 (M+H-69, Triazole)⁺.

### Example 16

### Preparation of 2-(4-Methoxyphenyl)-4-[1,2,4]triazol-1-yl-1,2,3,4-tetrahydro-quinoline of Formula (XVII)

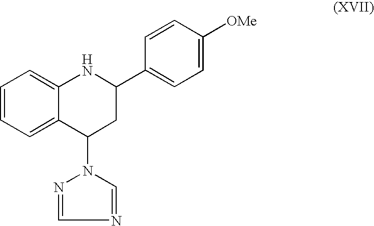

To a cooled (0-5° C.) solution of 1,2,4,-triazole (275 mg, 4 mmol) in dry acetonitrile (10 mL) is added a solution of thionyl chloride (120 mg, 1 mmol) in dry acetonitrile (1 mL) and the reaction is stirred at (0-5° C.) for 1 hr. The solution is filtered and added dropwise to solution of 2-(4-methoxy phenyl)-1,2,3,4-tetrahydro-quinolin-4-ol (255 mg, 1 mmol) in dry acetonitrile (10 mL). The reaction mixture is stirred under nitrogen atmosphere at room temperature for overnight, then it is filtered to remove solid residues and concentrated. The residue is dissolved in chloroform. The organic solution is washed with brine, dried over Na₂SO₄, crude product is purified by column chromatography on alumina using hexane:ethyl acetate 75:25 v/v. Yield 140 mg (46%); mp 170-172° C.; ¹H NMR (CDCL₃) δ 2.26 (ddd, 1H, H-3e), 2.63 (d, 1H, H-3a), 3.80 (s, 3H, OCH₃), 4.08 (dd, 1H, H-2), 4.25 (brs, 1H, NH), 5.51 (t, 1H, H-4), 6.68 (d, 1H, H-8), 6.74 (t, 1H, H-6), 6.86-7.26 (m, 6H, H-5, H-7, H-2′, H-3′, H-5′ and H-6′), 7.80 (s, 1H, H-3″), 8.02 (s, 1H, H-5″).

### Example 17

### Preparation of 2-(4-Chlorophenyl)-4-[1,2,4]triazol-1-yl-1,2,3,4-tetrahydro-quinoline of Formula (XVIII)

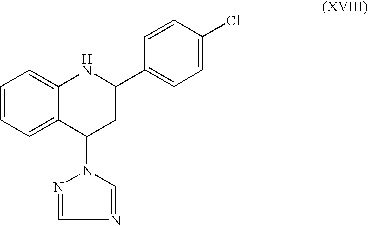

To a cooled (0-5° C.) solution of 1,2,4,-triazole (275 mg, 4 mmol) in dry acetonitrile (10 mL) is added a solution of thionyl chloride (120 mg, 1 mmol) in dry acetonitrile (1 mL) and the reaction is stirred at (0-5° C.) for 1 hr. The solution is filtered and added dropwise to solution of 2-(4-chloro phenyl)-1, 2, 3,4-tetrahydro-quinolin-4-ol (260 mg, 1 mmol) in dry acetonitrile (10 mL). The reaction mixture is stirred under nitrogen atmosphere at room temperature for overnight, then it is filtered to remove solid residues and concentrated. The residue is dissolved in chloroform. The organic solution is washed with brine, dried over Na₂SO₄, crude product is purified by column chromatography on alumina using hexane:ethyl acetate 75:25 v/v. Yield 110 mg (35%); mp 150-152° C.

### Example 18

### Preparation of 2-(4-Bromophenyl)-4-[1,2,4]triazol-1-yl-1,2,3,4-tetrahydro-quinoline of Formula (XIX)

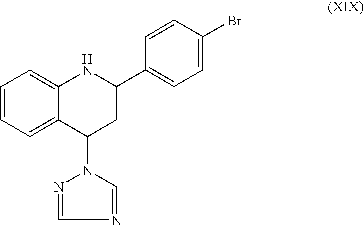

To a cooled (0-5° C.) solution of 1,2,4,-triazole (275 mg, 4 mmol) in dry acetonitrile (10 mL) is added a solution of thionyl chloride (120 mg, 1 mmol) in dry acetonitrile (1 mL) and the reaction is stirred at (0-5° C.) for 1 hr. The solution is filtered and added dropwise to solution of 2-(4-bromophenyl)-1,2,3,4-tetrahydro-quinolin-4-ol (304 mg, 1 mmol) in dry acetonitrile (10 mL). The reaction mixture is stirred under nitrogen atmosphere at room temperature for overnight, it is filtered to remove solid residues and concentrated. The residue is dissolved in chloroform. The organic solution is washed with brine, dried over Na₂SO₄, crude product is purified by column chromatography on alumina using hexane:ethyl acetate 75:25 v/v. Yield 140 mg (40%); mp 158-160° C.; ¹H NMR (CDCL₃) δ 2.23 (ddd, 1H, H-3e), 2.63 (d, 1H, H-3a), 4.08 (dd, 111, H-2), 4.27 (brs, 1H, NH), 5.51 (t, 1H, H-4), 6.69 (d, 1H, H-8), 6.77 (t, 1-H, H-6), 7.11 (d, H-5), 7.20-7.48 (m, 5H, H-7, H-2′, H-3′, H-5′ and H-6′), 7.79 (s, 1H, H-3″), 8.03 (s, 1H, H-5″).

### Example 19

### Preparation of 4-(4-[1,2,4]Triazol 2,3,4-tetrahydro-quinolin-2-yl)-benzonitrile of Formula (XX)

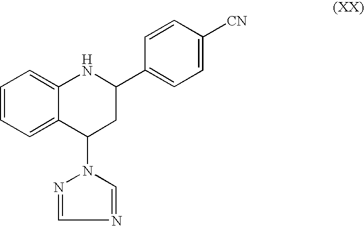

To a cooled (0-5° C.) solution of 1,2,4,-triazole (275 mg, 4 mmol) in dry acetonitrile (10 mL) is added a solution of thionyl chloride (120 mg, 1 mmol) in dry acetonitrile (1 mL) and the reaction is stirred at (0-5° C.) for 1 hr. The solution is filtered and added dropwise to solution of 4-(4-hydroxy-1,2,3,4-tetrahydroquinolin-2-yl)-benzonitrile (250 mg, 1 mmol) in dry acetonitrile (10 mL). The reaction mixture is stirred under nitrogen atmosphere at room temperature for overnight, it is filtered to remove solid residues and concentrated. The residue is dissolved in chloroform. The organic solution is washed with brine, dried over Na₂SO₄, crude product is purified by column chromatography on alumina using hexane: ethyl acetate 85:15 v/v. Yield 120 mg (40%); mp 170-172° C.; ¹H NMR (DMSO-d₆) δ 2.26 (m, 1H, H-3e), 2.48 (d, 1H, H-3a), 4.57 (dd, 1H, H-2), 5.52 (t, 1H, H-4), 6.27-7.85 (m, 8H, H-5, H-6, H-7, H-8, H-2′, H-3′, H-5′ and H-6′), 8.00 (s, 1H, H-3″), 8.40 (s, 1H, H-5″).

### Example 20

### Preparation of 2-(4-Fluorophenyl)-4-[1,2,4]triazol-1-yl-1,2,3,4-tetrahydro-quinoline of Formula (XXI)

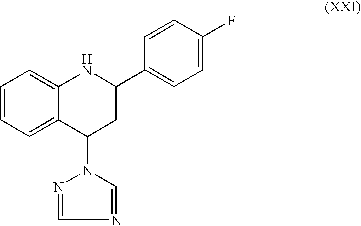

To a cooled (0-5° C.) solution of 1,2,4,-triazole (275 mg, 4 mmol) in dry acetonitrile (10 mL) is added a solution of thionyl chloride (120 mg, 1 mmol) in dry acetonitrile (1 mL) and the reaction is stirred at (0-5° C.) for 1 hr. The reaction mixture is filtered and added dropwise to solution of 2-(4-fluorophenyl) —1,2,3,4-tetrahydro-quinolin-4-ol (243 mg, 1 mmol) in dry acetonitrile (10 mL). The reaction mixture is stirred under nitrogen atmosphere at room temperature for overnight, then it is filtered to remove solid residues and concentrated. The residue is dissolved in chloroform. The organic solution is washed with brine, dried over Na₂SO₄, crude product is purified by column chromatography on alumina using hexane:ethyl acetate 75:25 v/v. Yield 160 mg (56%); mp 184-186° C.; ¹H NMR (CDCL₃) δ 2.25 (ddd, 1H, H-3e), 2.63 (dd, 1H, H-3a), 4.12 (dd, 1H, H-2), 4.26 (brs, 1H, NH), 5.51 (t, 1H, H-4), 6.69 (d, 1H, H-8), 6.77 (t, 1-H, H-6), 7.01-7.33 (m, 6H, H-5, H-7, H-2′, H-3′, H-5′ and H-6′), 7.80 (s, 1H, H-3″), 8.03 (s, 1H, H-5″). MS m/z 295 (M+H)⁺, 226 (M+H-69, Triazole)⁺.

### Example 21

### Preparation of 2-Phenyl-3-((pyridin-4-yl)methyl)quinolin-4 (1H)-one of Formula (XXII)

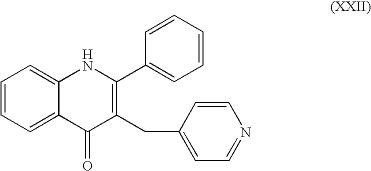

An aqueous sodium hydroxide solution (6N, 2 mL) is added to a solution of 2-phenyl-2,3-dihydroquinolin-4(1H)-one (223 mg, 1 mmol) in ethanol (5 mL). Pyridine-4-carboxaldehyde (126 mg, 1.2 mmol) is then added to the reaction mixture, which is stirred at room temperature for 24 hours. Subsequently, it is evaporated under reduced pressure. The residue is dissolved into chloroform and the organic layer washed with water, dried over Na₂SO₄ and evaporated to dryness. Crude product further purified by column chromatography on silica gel using ethyl acetate:acetone (97:03 v/v) as eluent. Yield 130 mg (42%); mp 222-224° C.; IR (KBR) 3435, 1627 cm⁻¹; ¹³C NMR (DMSO-d₆) δ 30.84 (CH₂), 116.04 (C-8), 118.40 (C-6), 123.16 (C-3), 123.39 (C-2″ and C-6″), 123.76 (C-4′), 125.09 (C-4a), 128.61 (C-2′ and C-6′), 128.66 (C-3′ and C-5′), 129.67 (C-5), 131.73 (C-7), 134.58 (C-1′), 139.65 (C-2), 149.16 (C-3″ and C-5″), 149.64 (C-1″), 150.49 (C-8a), 176.21 (C═O).

### Example 22

### Preparation of 2-(p-Tolyl)-3-((pyridin-4-yl)methyl)quinolin-4 (1H)-one of Formula (XXIII)

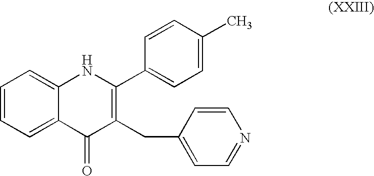

An aqueous sodium hydroxide solution (6N, 2 mL) is added to a solution of 2-(4-methylphenyl)-2,3-dihydroquinolin-4(1H)-one (237 mg, 1 mmol) in ethanol (5 mL). Pyridine-4-carboxaldehyde (126 mg, 1.2 mmol) is then added to the reaction mixture, which is stirred at room temperature for 24 hours. Subsequently, it is evaporated under reduced pressure. The residue is dissolved into chloroform and the organic layer washed with water, dried over Na₂SO₄ and evaporated to dryness. Crude product further purified by column chromatography on silica gel using ethyl acetate:acetone (97:03 v/v) as eluent. Yield 160 mg (49%); mp 264-266° C.; IR (KBR) 3403, 1623 cm⁻¹; ¹H NMR (DMSO-d₆) δ: 2.37 (s, 3H, CH₃), 3.70 (s, 2H, CH₂), 6.96 (d, 2H, H-2″ and H-6″), 7.31-7.64 (m, 7H, H-6, H-7, H-8, H-2′, H-3′, H-5′ and H-6′), 8.08 (d, 1H, H-5), 8.29 (d, 2H, H-3″ and H-5″), 11.74 (s, 1H, NH); MS m/z 327 (M+H)⁺

### Example 23

### Preparation of 2-(4-Methoxy phenyl)-3-((pyridin-4-yl)methyl)quinolin-4(1H)-one of Formula (XXIV)

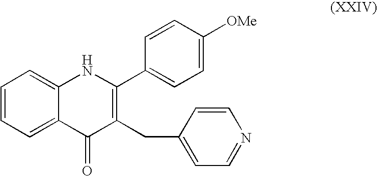

An aqueous sodium hydroxide solution (6N, 2 mL) is added to a solution of 2-(4-methoxyphenyl)-2,3-dihydroquinolin-4(1H)-one (253 mg, 1 mmol) in ethanol (5 mL). Pyridine-4-carboxaldehyde (126 mg, 1.2 mmol) is then added to the reaction mixture, which is stirred at room temperature for 24 hours. Subsequently, it is evaporated under reduced pressure. The residue is dissolved into chloroform and the organic layer is washed with water, dried over Na₂SO₄ and evaporated to dryness. Crude product further purified by column chromatography on silica gel using ethyl acetate:acetone (97:03 v/v) as eluent. Yield 130 mg (37%); mp 246-248° C.; MS m/z 343 (M+H)⁺.

### Example 24

### Preparation of 2-(4-Chlorophenyl)-3-((pyridin-4-yl)methyl)quinolin-4(1H)-one of Formula (XXV)

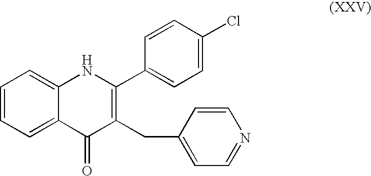

An aqueous sodium hydroxide solution (6N, 2 mL) is added to a solution of 2-(4-chlorophenyl)-2,3-dihydroquinolin-4(1H)-one (257 mg, 1 mmol) in ethanol (5 mL). Pyridine-4-carboxaldehyde (126 mg, 1.2 mmol) is then added to the reaction mixture, which is stirred at room temperature for 24 hours. Subsequently, it is evaporated under reduced pressure. The residue is dissolved into chloroform and the organic layer is washed with water, dried over Na₂SO₄ and evaporated to dryness. Crude product further purified by column chromatography on silica gel using ethyl acetate:acetone (97:03 v/v) as eluent. Yield 170 mg (49%); mp 232-234° C.; IR (KBR) 3444, 1623 cm⁻¹; ¹H NMR (DMSO-d₆) δ: 3.71 (s, 2H, CH₂), 6.94 (d, 2H, H-2″and H-6″), 7.24-7.59 (m, 7H, H-6, H-7, H-8, H-2′, H-3′, H-5′, H-6′), 8.13 (d, 1H, H-5), 8.30 (d, 2H, H-3″ and H-5″), 11.73 (s, 1H, NH, D₂O exchangeable); MS m/z 347 (M+H)⁺.

### Example 25

### Preparation of 2-(4-Bromophenyl)-3-((pyridin-4-yl)methyl)quinolin-4(1H)-one of Formula (XXVI)

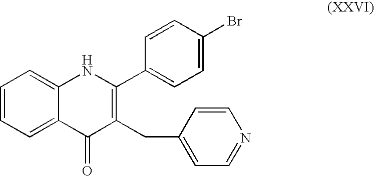

An aqueous sodium hydroxide solution (6N, 2 mL) is added to a solution of 2-(4-bromophenyl)-2,3-dihydroquinolin-4(1H)-one (302 mg, 1 mmol) in ethanol (5 mL). Pyridine-4-carboxaldehyde (126 mg, 1.2 mmol) is then added to the reaction mixture, which is stirred at room temperature for 24 hours. Subsequently, it is evaporated under reduced pressure. The residue is dissolved into chloroform and the organic layer is washed with water, dried over Na₂SO₄ and evaporated to dryness. Crude product further purified by column chromatography on silica gel using ethyl acetate:acetone (97:03 v/v) as eluent. Yield 180 mg (46%); mp 232-234° C.; IR (KBR) 3436, 1624 cm⁻¹; ¹H NMR (DMSO-d₆) δ: 3.82 (s, 2H, CH₂), 7.01 (d, 2H, H-2″and H-6″), 7.21-7.77 (m, 7H, H-6, H-7, H-8, H-2′, H-3′, H-5′, H-6′), 8.20-8.41 (m, 3H, H-5, H-3″ and H-5″), 11.50 (brs, 1H, NH).

### Example 26

### Preparation of 4-(4-oxo-3-((pyridin-4-yl)methylene)quinolin-2-yl)benzonitrile of Formula (XXVII)

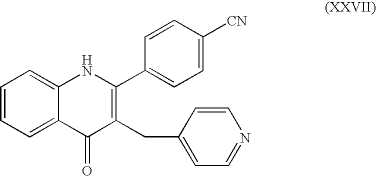

An aqueous sodium hydroxide solution (6N, 2 mL) is added to a solution of 4-(4-oxo-1,2,3,4-tetrahydroquinolin-2-yl)-benzonitrile (248 mg, 1 mmol) in ethanol (5 mL). Pyridine-4-carboxaldehyde (126 mg, 1.2 mmol) is then added to the reaction mixture, which is stirred at room temperature for 24 hours. Subsequently, it is evaporated under reduced pressure. The residue is dissolved into chloroform and the organic layer is washed with water, dried over Na₂SO₄ and evaporated to dryness. Crude product further purified by column chromatography on silica gel using ethyl acetate:acetone (97:03 v/v) as eluent. Yield 100 mg (29%); mp 154-156° C.; ¹H NMR (DMSO-d₆) δ: 3.68 (s, 2H, CH₂), 6.96 (d, 2H, H-2″and H-6″, J=5.7 Hz), 7.32-8.01 (m, 7H, H-6, H-7, H-8, H-2′, H-3′, H-5′ and H-6′), 8.12 (d, 1H, H-5, J=7.3 Hz), 8.29 (d, 2H, H-3″ and H-5″, J=5.7 Hz), 11.91 (s, 1H, NH); MS m/z 338 (M+H)⁺.

### Example 27

### Preparation of 2-(4-Fluorophenyl)-3-((pyridin-4-yl)methyl)quinolin-4(1H)-one of Formula (XXVIII)

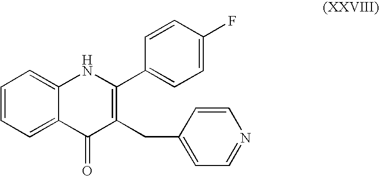

An aqueous sodium hydroxide solution (6N, 2 mL) is added to a solution 2-(4-fluorophenyl)-2,3-dihydroquinolin-4(1H)-one (241 mg, 1 mmol) in ethanol (5 mL). Pyridine-4-carboxaldehyde (126 mg, 1.2 mmol) is then added to the reaction mixture, which is stirred at room temperature for 24 hours. Subsequently, it is evaporated under reduced pressure. The residue is dissolved into chloroform and the organic layer is washed with water, dried over Na₂SO₄ and evaporated to dryness. Crude product further purified by column chromatography on silica gel using ethyl acetate:acetone (97:03 v/v) as eluent. Yield 120 mg (36%); mp 144-146° C.; ¹H NMR (DMSO-d₆) δ: 3.70 (s, 2H, CH₂), 6.96 (d, 2H, H-2″and H-6″), 7.30-7.69 (m, 7H, H-6, H-7, H-8, H-2′, H-3′, H-5′ and H-6′), 8.12 (d, 1H, H-5), 8.30 (d, 2H, H-3″ and H-5″), 11.81 (s, 1H, NH); MS m/z 331 (M+H)⁺.

### Example 28

### Preparation of 6,8-Dibromo-2-phenyl-3-((pyridin-4-yl)methyl)quinolin-4(1H)-one of Formula (XXIX)

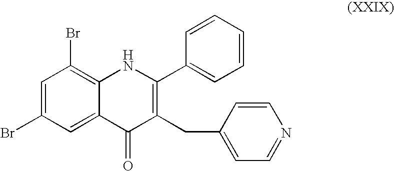

6,8-Dibromo-2-phenyl-2,3-dihydroquinolin-4(1H)-one (760 mg, 2 mmol) and pyridine-4-carboxaldehyde (215 mg, 2 mmol) are heated at 130° C. in the presence of few drops of piperidine for 2 hr. Then chloroform (30 mL) is added to the cooled reaction mixture and the organic layer is washed twice with water (2×20 mL), the residue is purified by column chromatography on silica gel with hexane:ethyl acetate 60:40 v/v. Yield 140 mg (15%); nip 190-192° C.; IR (KBR) 3376, 1617 cm⁻¹; ¹H NMR (CDCL₃) δ 3.9 (s, 2H, CH₂), 6.9-7.6 (m, 7H, H-2′, H-3′, H-4′, H-5′, H-6′, H-2″ and H-6″), 8.0 (s, 1H, H-7), 8.4 (d, 2H, H-3″ and H-5″), 8.5 (s, 1H, H-5).

### Example 29

### Preparation of 6,8-Dibromo-2-(p-tolyl)-3-((pyridin-4-yl)methyl)quinolin-4(1H)-one of Formula (XXX)

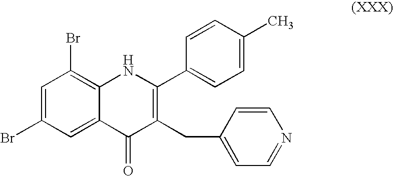

6,8-Dibromo-2-(p-tolyl)-2,3-dihydroquinolin-4(1H)-one (790 mg, 2 mmol) and pyridine-4-carboxaldehyde (215 mg, 2 mmol) are heated at 130° C. in the presence of few drops of piperidine for 2 hr. Then chloroform (30 mL) is added to the cooled reaction mixture and the organic layer is washed twice with water (2×20 mL), the residue is purified by column chromatography on silica gel with hexane:ethyl acetate 60:40 v/v. Yield 240 mg (25%); mp 206-208° C.; IR (KBR) 3374, 1620 cm⁻¹; ¹H NMR (CDCL₃) δ 2.4 (s, 3H, CH₃), 3.9 (s, 2H, CH₂), 6.9-7.4 (m, 6H, H-2′, H-3′, H-5′, H-6′, H-2″and H-6″), 8.0 (s, 1H, H-7), 8.4 (d, 2H, H-3″ and H-5″), 8.5 (s, 1H, H-5).

### Example 30

### Preparation of 6,8-Dibromo-2-(4-Methoxyphenyl)-3-((pyridin-4-yl)methyl)quinolin-4 (113)-one of Formula (XXXI)

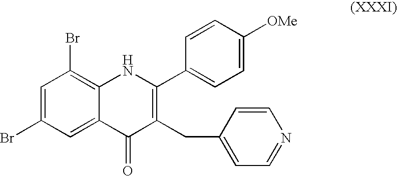

6,8-Dibromo-2-(4-methoxyphenyl)-2,3-dihydroquinolin-4(1H)-one (820 mg, 2 mmol) and pyridine-4-carboxaldehyde (215 mg, 2 mmol) are heated at 130° C. in the presence of few drops of piperidine for 2 hr. Then chloroform (30 mL) is added to the cooled reaction mixture and the organic layer is washed twice with water (2×20 mL), the residue is purified by column chromatography on silica gel with hexane:ethyl acetate 60:40 v/v. Yield 150 mg (15%); mp 194-196° C.; IR (KBR) 2924, 1608 cm⁻¹; ¹H NMR (CDCL₃) δ 3.9 (s, 2H, CH₂), 6.9-7.4 (m, 6H, H-2′, H-3′, H-5′, H-6′, H-2″and H-6″), 8.0 (s, 1H, H-7), 8.4 (d, 2H, H-3″ and H-5″), 8.5 (s, 1H, H-5).

### Example 31

### Preparation of 6,8-Dibromo-2-(4-Chloro phenyl)-3-((pyridin-4-yl)methyl)quinolin-4(1H)-one of Formula (XXXII)

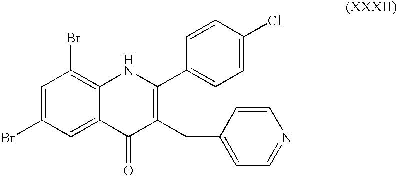

6,8-Dibromo-2-(4-chlorophenyl)-2,3-dihydroquinolin-4(1H)-one (830 mg, 2 mmol) and pyridine-4-carboxaldehyde (215 mg, 2 mmol) are heated at 130° C. in the presence of few drops of piperidine for 2 hr. Then chloroform (30 mL) is added to the cooled reaction mixture and the organic layer is washed twice with water (2×20 mL), the residue is purified by column chromatography on silica gel with hexane:ethyl acetate 60:40 v/v. Yield 300 mg (30%); mp 198-200° C.; IR (KBR) 2918, 1608 cm⁻¹; ¹H NMR (CDCL₃) δ 3.9 (s, 2H, CH₂), 6.9 (d, 2H, H-2″and H-6″), 7.3-7.6 (m, 4H, H-2′, H-3′, H-5′ and H-6′), 8.0 (d, 1H, H-7), 8.4 (d, 2H, H-3″ and H-5″), 8.5 (s, 1H, H-5).

### Example 32

### Aromatase Inhibition Assay

(This assay has been performed at Pharmaceutical Chemistry laboratories, Saarland University, Germany in collaboration with Prof R. W. Hartmann. These laboratories are governed by institutional ethical committee and European union human tissue regulations)

The reaction mixture containing [1β-H³] androstenedione (0.08 μCi, 15 nM), unlabeled testosterone (500 nM), the NADPH generating system, the inhibitor (500 nM) and phosphate buffer (0.05M, pH7.4) is preincubated for 5 min at 30° C. Microsomal protein (1 mg, from human placental tissue) is added to the start the reaction. After incubation for 15 min at 30° C., the reaction is stopped by adding 200 μL of a cold HgCl₂ solution (1 mM). After addition of 200 μL of an aqueous dextran coated charcoal suspension (2%), the vials are shaken then centrifuged to separate the charcoal adsorbed steroids. Aliquots of the supernatant were assayed for ³H₂O by counting in a scintillation mixture in a scintillation counter.

TABLE 3
Aromatase inhibition activity of quinolines
in human placental microsomes

| Compound | % inhibition* |
|---|---|
| 6a | 61.77 |
| 6b | 67.43 |
| 6c | 53.07 |
| 6d | 87.37 |
| 6e | 89.70 |
| 6f | 84.33 |
| 6g | 80.50 |
| 6h | 11.17 |
| 6i | 13.60 |
| 6j | 12.80 |
| 6k | 41.90 |
| 6l | 43.90 |
| 6m | 36.07 |
| 6n | 19.50 |
| 10a | 07.20 |
| 10b | 03.10 |
| 10c | 00.70 |
| 10d | 13.70 |
| 10e | 08.20 |
| 10f | 01.80 |
| 10g | 11.80 |
| 10h | 27.30 |
| 10i | 22.30 |
| 10j | 40.50 |
| 10k | 55.70 |

1. A compound of formula (I) 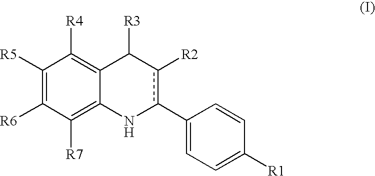
wherein,
dotted line represents optional double bond;
R1 is —H, halo, —(C₁-C₃)alkyl, —O(C₁-C₃)alkyl or —CN; R2 is —H or 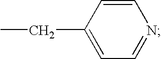
R3 comprises —O—, heterocycle moiety, preferably of ring size 5-6; wherein the ring is selected from imidazole, triazole, tetrazole and/or pyridine; wherein further the said ring comprises substituents selected from —H, —(C₁-C₃)alkyl, —O(C₁-C₃)alkyl, F, —CF₃, —NH₂, —N(CH₃), —N(CH₃)₂, —SH, —SCH₃, —SCH₂CH₃ and/or combinations thereof;
R4, R5, R6, R7 are, independently a moiety selected from the group consisting of —H, halo, —(C₁-C₃)alkyl, —O(C₁-C₃)alkyl or —CF₃;
or a pharmaceutically acceptable salt or derivative thereof.

2. The compound as claimed in claim 1, wherein R1 is preferably selected from the group comprising —H, Cl, Br, F, —CH₃, —OCH₃ or —CN.

3. The compound as claimed in claim 1, wherein R3 is preferably selected from the group comprising

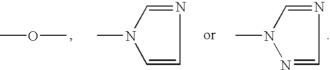

4. The compound as claimed in claim 1, wherein R3 is heterocyclic ring, preferably a five membered ring comprising 1-3 nitrogen atoms in the ring.

5. The compound as claimed in claim 1, wherein R4, R5, R6 and R7 are preferably selected from H or Br.

6. The compound as claimed in claim 1, which is selected from the group comprising:
2-Phenyl-4-imidazol-1-yl-1,2,3,4-tetrahydro-quinoline;
2-(p-Tolyl)-4-imidazol-1-yl-1,2,3,4-tetrahydro-quinoline;
2-(4-Methoxy phenyl)-4-imidazol-1-yl-1,2,3,4-tetrahydro-quinoline;
2-(4-Chloro phenyl)-4-imidazol-1-yl-1,2,3,4-tetrahydro-quinoline;
2-(4-Bromo phenyl)-4-imidazol-1-yl-1,2,3,4-tetrahydro-quinoline;
4-(4-Imidazol-1-yl-1,2,3,4-tetrahydro-quinolin-2-yl)-benzonitrile;
2-(4-Fluoro phenyl)-4-imidazol-1-yl-1,2,3,4-tetrahydro-quinoline;
2-Phenyl-4-[1,2,4]triazol-1-yl-1,2,3,4-tetrahydro-quinoline;
2-(p-Tolyl)-4-[1,2,4]triazol-1-yl-1,2,3,4-tetrahydro-quinoline;
2-(4-Methoxy phenyl)-4-[1,2,4]triazol-1-yl-1,2,3,4-tetrahydro-quinoline;
2-(4-Chloro phenyl)-4-[1,2,4]triazol-1-yl-1,2,3,4-tetrahydro-quinoline;
2-(4-Bromo phenyl)-4-[1,2,4]triazol-1-yl-1,2,3,4-tetrahydro-quinoline;
4-(4-[1,2,4]Triazol-1-yl-1,2,3,4-tetrahydro-quinolin-2-yl)-benzonitrile;
2-(4-Fluoro phenyl)-4-[1,2,4]triazol-1-yl-1,2,3,4-tetrahydro-quinoline;
2-Phenyl-3-((pyridin-4-yl)methyl)quinolin-4(1H)-one;
2-(p-Tolyl)-3-((pyridin-4-yl)methyl)quinolin-4(1H)-one;
2-(4-Methoxy phenyl)-3-((pyridin-4-yl)methyl)quinolin-4(1H)-one;
2-(4-Chloro phenyl)-3-((pyridin-4-yl)methyl)quinolin-4(1H)-one;
2-(4-Bromo phenyl)-3-((pyridin-4-yl)methyl)quinolin-4(1H)-one;
4-(4-oxo-3-((pyridin-4-yl)methylene)quinolin-2-yl)benzonitrile;
2-(4-Fluoro phenyl)-3-((pyridin-4-yl)methyl)quinolin-4(1H)-one;
6,8-Dibromo-2-phenyl-3-((pyridin-4-yl)methyl)quinolin-4(1H)-one;
6,8-Dibromo-2-(p-tolyl)-3-((pyridin-4-yl)methyl)quinolin-4(1H)-one;
6,8-Dibromo-2-(4-Methoxy phenyl)-3-((pyridin-4-yl)methyl)quinolin-4(1H)-one;
6,8-Dibromo-2-(4-Chloro phenyl)-3-((pyridin-4-yl)methyl)quinolin-4(1H)-one;
a stereoisomer, a derivative and/or pharmaceutically acceptable salts thereof.

7. A process for the preparation of compound of formula (I): 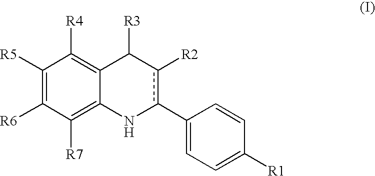
wherein,
dotted line represents optional double bond;
R1 is —H, halo, —(C₁-C₃)alkyl, —O(C₁-C₃)alkyl or —CN;
R2 is —H or —(4-Pyridyl methyl);
R3 comprises —O—, heterocycle moiety, preferably of ring size 5-6; wherein the ring is substituents selected from —H, —O(C₁-C₃)alkyl, F, —CF₃, —NH₂, —N(CH₃), —N(CH₃)₂, —SH, —SCH₃, —SCH₂CH₃ and/or combinations thereof;
R4, R5, R6, R7 are, independently a moiety selected from the group consisting of —H, —O(C₁-C₃)alkyl or —CF₃;
or a pharmaceutically acceptable salt or derivative thereof;
such as hereinbefore described in SCHEME 1, comprising the steps of:
(a) synthesising 2′-Amino chalcones by condensing 2′-Amino acetophenone and para substituted benzaldehydes;
(b) cyclising the 2′-Amino chalcones obtained in step (a) in presence of acid catalyst to prepare 2-Aryl-1,2,3,4-tetrahydro-4-quinolones;
(c) reducing the 2-Aryl-1,2,3,4-tetrahydro-4-quinolones of step (b) with NaBH₄ to give respective carbinols.
(d) further converting the respective carbinols to imidazole or triazole compounds by reacting the said carbinols with imidazole or triazole containing compounds.

8. A process for the preparation of compound of formula (I): 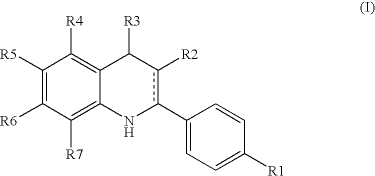
wherein,
dotted line represents optional double bond;
R1 is —H, halo, —(C₁-C₃)alkyl, —O(C₁-C₃)alkyl or —CN;
R2 is —H or —(4-Pyridyl methyl);
R3 comprises —O—, heterocycle moiety, preferably of ring size 5-6; wherein the ring is selected from imidazole, triazole, tetrazole and/or pyridine; wherein further the said ring comprises substituents selected from —H, —(C₁-C₃)alkyl, —O(C₁-C₃)alkyl, F, —CF₃, —NH₂, —N(CH₃), —N(CH₃)₂, —SH, —SCH₃, —SCH₂CH₃ and/or combinations thereof;
R4, R5, R6, R7 are, independently a moiety selected from the group consisting of —H, halo, —(C₁-C₃)alkyl, —O(C₁-C₃)alkyl or —CF₃;
or a pharmaceutically acceptable salt or derivative thereof;
such as hereinbefore described in SCHEME 2, comprising the steps of:
(a) synthesising 2′-Amino chalcones by condensing 2′-Amino acetophenone and para substituted benzaldehydes;
(b) cyclising the 2′-Amino chalcones obtained in step (a) in presence of acid catalyst to prepare 2-Aryl-1,2,3,4-tetrahydro-4-quinolones;
(c) condensing the 2-Aryl-1,2,3,4-tetrahydro-4-quinolones of step (b) with Pyridine-4-Carbaxaldehyde.

9. A pharmaceutical composition comprising a compound of formula (I) as claimed in claim 1, and a pharmaceutically acceptable carrier, diluent, excipent or solvate.

10. The compound of formula (I), as claimed in claim 1 used in the manufacture of medicament for treating or preventing cancer, particularly breast cancer, more particularly hormone dependent breast cancer.

11. A compound of formula (I) as substantially hereinbefore described and with reference to the foregoing examples.

12. A process for the preparation of compound of formula (I) as substantially hereinbefore described and with reference to the foregoing examples.

13. A pharmaceutical composition comprising compound of formula (I) as substantially hereinbefore described and with reference to the foregoing examples.
